(19) United States
(12) Patent Application Publication
(10) Pub. No.: US 2024/0150286 A1
(43) Pub. Date: May 9, 2024

(54) PEROXISOME PROLIFERATOR-ACTIVATED RECEPTOR GAMMA (PPARy) LIGANDS AND METHODS OF USE AS TREATMENTS FOR INSULIN RESISTANCE, OBESITY, FATTY LIVER DISEASE, AND TYPE 2 DIABETES

(71) Applicant: The Board of Regents of the University of Oklahoma, Norman, OK (US)

(72) Inventors: Venkateswararao Eeda, Oklahoma City, OK (US); Weidong Wang, Edmond, OK (US); Dan Wu, Moore, OK (US)

(21) Appl. No.: 18/383,708

(22) Filed: Oct. 25, 2023

Related U.S. Application Data
(60) Provisional application No. 63/419,049, filed on Oct. 25, 2022.

(51) Int. Cl. C07D 209/18; A61P 1/16; A61P 3/04; A61P 3/10

(52) U.S. Cl. CPC C07D 209/18; A61P 1/16; A61P 3/04; A61P 3/10

(57) ABSTRACT

Compounds and methods for treating insulin resistance, obesity, Type 2 diabetes, Nonalcoholic fatty liver disease (NAFLD), and peroxisome proliferator-activated receptor gamma (PPARy) serine 273 phosphorylation in subjects in need of such therapy.

### CROSS-REFERENCE TO RELATED APPLICATIONS

The present application claims the benefit of U.S. Provisional Patent Application Ser. No. 63/419,049 filed Oct. 25, 2022, the disclosure of which is hereby expressly incorporated by reference in its entirety.

### STATEMENT REGARDING FEDERALLY SPONSORED RESEARCH AND DEVELOPMENT

This invention was made with government support under Grant Numbers R01DK116017 and R01DK128848 awarded by the National Institutes of Health. The government has certain rights in the invention.

### BACKGROUND

The nuclear receptor peroxisome proliferator-activated receptor gamma (PPARγ) plays an important role in the regulation of insulin sensitivity, glucose and lipid homeostasis, and adipogenesis. It is therefore an important therapeutic target for insulin resistance and diabetes treatment. Indeed, the commonly prescribed anti-diabetic thiazolidinedione (TZD) drugs (also known as glitazones) such as rosiglitazone (Rosi) act as full agonists of PPARγ to improve insulin sensitization. However, TZD drugs are associated with several serious side effects including water retention, adiposity, weight gain, and congestive heart failure, which are believed to be caused by the full PPARγ agonism of TZDs, thereby severely hindering their clinical use. Therefore, PPARγ agonists that maintain insulin sensitizing effect but minimize or eliminate TZD-related side effects would be ideal for the treatment of insulin resistance.

Recent studies indicate that the insulin sensitizing effect of TZDs is mediated by their inhibition of PPARγ phosphorylation at the serine 273 position (S273) of the protein, independent of their full agonism on PPARγ. In obesity, PPARγ is phosphorylated at S273 by obesity-activated protein kinases cyclin-dependent kinase 5 (CDKS) and extracellular signal-regulated kinase (ERK). Phosphorylation of PPARγ at S273 induces the dysregulation of a number of genes critical for insulin sensitivity, such as insulin-sensitizing adipokine adiponectin, leading to insulin resistance. TZDs inhibit the CDK5- or ERK-induced phosphorylation of PPARγ at the s273, thereby reversing insulin resistance in obesity. Importantly, the inhibitory activity on S273 phosphorylation by TZDs is independent of the TZD-associated deleterious side effects, thus revealing the blockade of S273 dephosphorylation as a critical means of decoupling the insulin sensitizing effect from the full agonism-associated side effects. Several small molecule PPARγligands were recently reported to inhibit PPARγ S273 phosphorylation and harbor insulin sensitizing and anti-diabetic activity without TZD-associated side effects, but are of low potency (Choi J H, Banks A S, Kamenecka T M, Busby S A, Chalmers M J, Kumar N, et al. Antidiabetic actions of a non-agonist PPARgamma ligand blocking Cdk5-mediated phosphorylation. Nature. 2011;477(7365):477-81; and Choi S-S, Kim E S, Koh M, Lee S-J, Lim D, Yang Y R, et al. A novel non-agonist peroxisome proliferator-activated receptor γ (PPARγ) ligand UHC1 blocks PPARγ phosphorylation by cyclin-dependent kinase 5 (CDK5) and improves insulin sensitivity. Journal of Biological Chemistry. 2014;289(38):26618-29).

Body fat (adipose tissue) exists in different colors and functions. Beige (a.k.a., brite) and brown fat cells (adipocytes) metabolize lipids to generate heat, while white adipocytes store energy as triglycerides. Studies have shown that beige and brown fat cells counteract obesity and metabolic diseases.

PPARγ ligand TZDs are known to have the advantageous property of inducing the conversion of energy-storing white adipocytes into energy-burning beige or brite adipocytes in white adipose tissues (WAT), a process commonly referred as browning. In studies comparing TZDs and certain partial agonists it was observed that the TZDs, but not the partial agonists, were capable of promoting the browning effect, which led to the interpretation that full agonism of PPARγ is required for the browning effect. Thus, it has heretofore been unknown whether a partial PPARγ agonist with the ability to inhibit PPARγ phosphorylation could be capable of inducing the browning effect.

It is to providing compounds that bind to PPARγ with high-affinity and specificity and improve insulin sensitivity and the browning effect without TZD-associated side effects that the present disclosure is directed.

### BRIEF DESCRIPTION OF THE DRAWINGS

Several embodiments of the present disclosure are hereby illustrated in the appended drawings. It is to be noted, however, that the appended drawings only illustrate several typical embodiments and are therefore not intended to be considered limiting of the scope of the disclosure. The figures are not necessarily to scale and certain features and certain views of the figures may be shown as exaggerated in scale or in schematic in the interest of clarity and conciseness. The patent or application file contains at least one drawing executed in color. Copies of this patent or patent application publication with color drawing(s) will be provided by the Office upon request and payment of the necessary fee.

FIG. 1 shows that WO95E is a high affinity partial agonist of PPARγ. (A) Chemical structure of WO95E. (B) Binding affinity of WO95E to the ligand binding domain of PPARγ in in vitro competition binding assay. Florescence polarization value (mP) was measured at 485 nm (excitation) and at 535 nm (emission). The data shown are representative of 3 independent experiments. (C) Transactivation activity of compounds in HEK293 cells transfected with PPARγ2 and 3×PPRE-luc reporter. Transactivation activity was presented as fold change with that of Rosi designated as 1. The data shown are representative of 3 independent experiments. (D) Oil Red O staining in 3T3-L1 cells differentiated with differentiation media in the presence of compounds at 10 μM. (E) Relative mRNA expression levels of adipogenic genes in 3T3-L1 differentiated with differentiation media in the presence of compounds at 10 μM by qRT-PCT. The results are expressed as fold change and are representative of 3 independent experiments. Data are the mean±SEM. *P<0.05, **P<0.01, and ***P<0.001.

FIG. 2 shows that WO95E inhibits PPARγ S273 phosphorylation. (A) Phosphorylation of PPARγ S273 in 3T3-L1 differentiated adipocytes treated with TNFα 10 ng/ml for 1 h in the presence of compounds at 10 μM. pSer273 of PPARγ was detected by Western blotting using PPARγ pSer273-specific antibody. The data shown are representative of 3 independent experiments. (B) Quantification of Phosphorylation of PPARγ S273. Data are the mean±SEM. *P<0.05, and ***P<0.001 compared to DMSO control in the presence of TNFα. (C) Relative mRNA expression levels of pSer273-associated genes in 3T3-L1 differentiated with differentiation media in the presence of compounds at 10 μM by qRT-PCR. The results are expressed as fold change and are representative of 3 independent experiments. Data are the mean±SEM. *P<0.05, **P<0.01, and ***P<0.001. (D) Docking simulation of the PPARγ LBD:WO95E complex. WO95E is in red. (E) Schematic representation of atomic interaction between PPARγ LBD and WO95E. Hydrogen bonds were shown in brown dashed lines with donor-acceptance distances in angstroms. (F-G) Transactivation activity of compounds in HEK293 cells transfected with PPARγ2 and 3×PPRE-luc reporter, in the presence and absence of GW9662 (5 μM). Both Rosi (F) and WO95E (G) were tested at 10 μM. Transactivation activity was presented as fold change with that of DMSO as 1. The data shown are representative of 3 independent experiments. Data are the mean±SEM. *P<0.05. (H) Transactivation activities shown in (F-G) were plotted as ratio of a compound in the presence of GW9662 over that in the absence of GW9662. Data are the mean±SEM. **P<0.01 compared to DMSO control group.

FIG. 3 shows that WO95E improves glucose tolerance and insulin sensitivity in DIO mice. (A-A′) Glucose tolerance test performed for mice treated with WO95E (n=8) or vehicle (n=7) for 2 weeks. Blood glucose levels (A) measured at indicated time points after intraperitoneal injection of glucose (1.5 g/kg body weight) following 6-h fasting and the AUC (area under the curve, A′). (B-B′) Insulin tolerance test performed for mice treated with WO95E (n=8) or vehicle (n=7) for 2.5 weeks. Blood glucose levels (B) normalized to basal level at indicated time points after intraperitoneal injection of insulin (1.2 IU/kg body weight) following 6-h fasting and the AUC (area under the curve, B′). (C) Plasma insulin levels at indicated time points after intraperitoneal injection of glucose (1.5 g/kg body weight) following 6-h fasting. (D) Plasma insulin levels after overnight fasting and 3-hour refeeding. (E) Serum free fatty acid level. (F) Serum total cholesterol level. (G-H) Phosphorylation status of PPARγ protein at S273 in iWAT (G) and in eWAT (H) determined by Western blotting. The data shown are representative of 3 independent experiments. (I) mRNA levels of known PPARγ phosphorylation-dependent genes determined by qRT-PCR. (J) mRNA levels of adipogenic and lipogenic genes determined by qRT-PCR. The results are expressed as fold change and are representative of 3 independent experiments. Data are the mean±SEM. #P<0.1, *P<0.05, **P<0.01, and ***P<0.001.

FIG. 4 shows that WO95E protects against obesity, decreases fat weight, and increases energy expenditure in DIO mice. (A) Body weight of DIO mice treated with WO95E (n=8) or vehicle (n=7). (B) Daily food intake, measured for three days during the 4^{th} week of treatment. (C) Fat mass. (D) Lean mass. E-F. eWAT adipocytes. (E) Representatives H&E staining of eWAT. (F) Average diameter of adipocytes (μM)/field and size distribution. (G-H) iWAT adipocytes. (G) Representatives H&E staining of iWAT. (H) Average diameter (μM)/field and size distribution. Arrows point to multilocular cells. (I-L) Metabolic cage analysis after 4-week treatment. (I-I′) Measurement of oxygen consumption levels. (J-J′) Measurement of CO₂ production levels. (K-K′) Energy expenditure levels. (L) Distance of activity (m/mouse). Data are the mean±SEM. *P<0.05, **P<0.01, and ***P<0.001.

FIG. 5 shows that WO95E promotes WAT browning. (A-B) mRNA levels of browning/thermogenic marker genes in iWAT (A) and in eWAT (B) from DIO mice treated with WO95E vs. vehicle. The results are expressed as fold change and are representative of 3 independent experiments. Data are the mean±SEM. *P<0.05, **P<0.01, and ***P<0.001. (C-D) Protein levels of UCP1 in iWAT (C) and eWAT (D) from DIO mice treated with WO95E vs. vehicle, assessed by Western blotting. The data shown are representative of 3 independent experiments. (E) Representative images of immunochemistry staining of UCP1 protein in iWAT from DIO mice treated with WO95E vs. vehicle. (F-G) mRNA levels of browning/thermogenic marker genes in adipocytes differentiated from wild-type iWAT (F) and eWAT (G) treated with DMSO or WO95E. The results are expressed as fold change and are representative of 3 independent experiments. Data are the mean±SEM. *P<0.05, **P<0.01, and ***P<0.001.

FIG. 6 shows that WO95E suppresses inflammation in adipose tissues in DIO mice. (A-A′) Representative images of H.E. staining of iWAT from DIO mice treated with WO95E or vehicle (A). Quantification of percentage of CLS area in iWAT (A′). (B-B′) Representative images of H.E. staining of eWAT from DIO mice treated with WO95E or vehicle (B). Quantification of percentage of CLS area in eWAT (B′). (C-D) mRNA levels of genes as pan-macrophage markers in iWAT (C) and eWAT (D). (E) mRNA levels of genes associated with M1 subtype in iWAT. (F) mRNA levels of genes associated with M2 subtype in iWAT. The results are the means of 3 replicate wells and are representative of 3 independent experiments for C-F. Data are the mean±SEM. *P<0.05, **P<0.01, and ***P<0.001.

FIG. 7 shows that WO95E improves liver steatosis in DIO mice. (A) Representative images of H&E staining of liver slides from mice fed normal chow diet, or fed HFD treated with WO95E or vehicle. (B) mRNA levels of lipogenic genes in livers from DIO mice treated with WO95E or vehicle. (C) mRNA levels of gluconeogenic genes in livers from DIO mice treated with WO95E or vehicle. The results are the means of 3 replicate wells and are representative of 3 independent experiments for B and C. Data are the mean±SEM. *P<0.05, **P<0.01, and ***P<0.001.

FIG. 8 shows that WO95E is devoid of common TZD-associated side effects. (A) PCV measurement in DIO mice treated with WO95E (n=8) or vehicle (n=7). (B) Heart weight of DIO mice treated with WO95E (n=8) or vehicle (n=7). (C-D) mRNA levels of heart failure/hypertrophy associated genes Myh7 (C) and Nppb (D) in hearts from DIO mice treated with WO95E or vehicle. (E-G) mRNA levels of bone density/formation-associated genes ColA1 (E), Bglap (F), and SPP1 (G) in bones from DIO mice treated with WO95E or vehicle. The results are the means of 3 replicate wells and are representative of 3 independent experiments for B and C. Data are the mean±SEM. *P<0.05, **P<0.01, and ***P<0.001.

### DETAILED DESCRIPTION

The present disclosure is directed to a class of compounds, including but not limited to the non-TZD indole derivative designated herein as WO95E, that possesses highly potent PPARγbinding affinity and specificity and inhibits PPARγ S273 phosphorylation, but with only mild PPARγ transactivation activity, and to methods of their use in treating, for example, insulin resistance, obesity, and Type 2 diabetes, NAFLD, and PPARγ S273 phosphorylation, and conditions related thereto, and for promoting conversion of white adipocytes to beige and/or brown adipocytes. The novel compounds increase insulin sensitivity (i.e., ameliorate insulin resistance) without causing significant adiposity, water retention, weight gain, or heart hypertrophy. In a non-limiting embodiment, the compounds (e.g., WO95E) protect against diet-induced obesity and increases the white-to-brown adipocyte conversion and energy expenditure. This is the first demonstration of a class of compounds that act as potent therapeutic agents in insulin sensitization and browning remodeling of white adipose tissues (WAT) and thus can be used for the treatment of T2D and obesity.

Briefly, in a non-limiting embodiment, the indole-based chemical 4′-((5-((3-hydroxybenzyl)carbamoyl)-2,3-dimethyl-1H-indol-1-yl)methyl)-[1,1′-biphenyl]-2-carboxylic acid, designated herein as WO95E, was tested for PPARγ binding and activity and for its effect on PPARγ phosphorylation. Diet-induced obese mice were administered WO95E for four weeks. Insulin sensitivity, glucose tolerance, body weight, fat tissue weight, adipocyte size, morphology, energy expenditure, and expression levels of genes involved in PPARγ activity, thermogenesis/browning, and TZD-related side effects were evaluated. Results indicated that WO95E binds to PPARγ with high affinity and acts as a PPARγ partial agonist. WO95E inhibits PPARγ phosphorylation and regulates PPARγ phosphorylation-dependent genes. WO95E ameliorates insulin resistance and glucose tolerance in mice of diet-induced obesity, with minimal TZD-related side effects.

It was also observed that WO95E promotes white-to-brown adipocyte conversion and energy expenditure and hence protects against diet-induced obesity. WO95E decreases the size of adipocytes and suppresses adipose tissue inflammation. WO95E also suppresses obesity-associated liver steatosis. Thus, WO95E improves insulin sensitivity and glucose homeostasis and promotes browning and energy expenditure by acting as a novel PPARγ phosphorylation inhibitor/partial agonist. These results demonstrate that this compound and others in its class can be used for the therapeutic treatment of, for example, insulin resistance, obesity, diabetes type 2, NAFLD, and PPARγ S273 phosphorylation, and conditions related thereto, and for promoting conversion of white adipocytes to beige and/or brown adipocytes.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application, including United States Provisional Patent Application Serial No. 63/419,049 filed Oct. 25, 2022, are herein expressly incorporated by reference in their entirety to the same extent as if each individual patent or publication was specifically and individually indicated to be incorporated by reference.

The following abbreviations may be used herein:
T1D, type 1 diabetes;
T2D, type 2 diabetes;
TZD, thiazolidinedione;
S273, serine 273 position;
PPARγ, peroxisome proliferator-activated receptor gamma;
WAT, white adipose tissue;
eWAT, epididymal white adipose tissue;
iWAT, inguinal white adipose tissue;
rosi, rosiglitazone;
GW9662, 2-Chloro-5-nitro-N-phenyl-benzamide;
DIO, diet-induced obesity;
DMSO, dimethylsulfoxicie;
ipGTT, Intraperitoneal glucose tolerance test;
ipITT, Intraperitoneal insulin tolerance test;
SCD-1, stearoly-coA desaturase-1;
ACC, acetyl coA-carboxylase;
G6PC, glucose-6-phosphatase catalytic subunit;
PEPCK, phosphoenolpyruvate carboxykinase;
PCV, packed-cell volume;
NAFLD, nonalcoholic fatty liver disease;
GADPH, glyceraldehyde 3 phosphate dehydrogenase;
RNA, Ribonucleic acid;
mRNA, messenger RNA;
DNA, Deoxyribonucleic acid;
ATP, adenosine triphosphate;
LBD, ligand binding domain;
mAb, monoclonal antibody;
TNFα, tumor necrosis factor alpha;
Tm, tunicamycin;
H&E, hematoxylin and eosin;
ER, endoplasmic reticulum;
STAT1, signal transducer and activator of transcription 1;
INS-1 cells, rat insulinoma cell line-1;
DIEA, N,N-diisopropylethylamine;
UPR, unfolded protein response;
SAR, structure-activity relationship;
EC₅₀, half maximal effective concentration;
qRT-PCR, quantitative reverse transcription polymerase chain reaction;
BFA, brefeldin A;
IRE1α, inositol-requiring protein 1α;
CHOP, C/EBP homologous protein;
PARP, Poly(ADP-ribose) polymerase;
Casp3, Caspase-3;
SR1664, CAS # 1338259-05-4;
UHC1, 4′ -((2,3-Dimethyl-5-(pyridine-3-ylmethylcarbo amyl)-1H-indol-1-yl)methyl)biphenyl-2-carboxylic acid;
eIF2α, eukaryotic translation initiator factor 2α;
TUNEL, Terminal deoxynucleotidyl transferase dUTP nick end labeling;
GSIS, Glucose Stimulated Insulin Secretion;
PERK, PKR (RNA-activated protein kinase)-like endoplasmic reticulum kinase;
XBP1, X-box binding protein 1;
ATF6, activating transcription factor 6;
ATF4, activating transcription factor 4;
FBS, fetal bovine serum;
PBS, phosphate-buffered saline;
PDX1, pancreatic and duodenal homeobox 1;
MafA, v-maf musculoaponeurotic fibrosarcoma oncogene family, protein A;
INS1, insulin 1;
INS2, insulin 2;
mAb, monoclonal antibody;
ALS, amyotrophic lateral sclerosis;
AD, Alzheimer's disease;
PSP, progressive supra nuclear palsy;
Halo, halogen;
Cl, chlorine;
F, fluorine;
Br, bromine;
I, iodine;
FBS, fetal bovine serum;
IBMX, 3-isobutyl-1-methylxanthine;
DMEM, Dulbecco's Modified Eagle Medium;
HEPES, (4-(2-hydroxyethyl)-1-piperazineethanesulfonic acid;
CT, cycle threshold;
ΔΔCt, delta delta CT method;
F, forward:
R, reverse;
ANOVA, analysis of variance;
GLM, generalized linear model;
SVF, stromal vascular fraction;
HFD, high-fat diet;
PVDF, polyvinylidene difluoride;
HRP, horseradish peroxidase;
HPLC, High pressure liquid chromatography;
DCM, dichloromethane;
DIPEA, diisopropylethylamine;
HATU, hexafluorophosphate azabenzotriazole tetramethyl uronium;
TFA, trifluoroacetic acid;
HBA, Hydroxybenzamide;
OMe, methoxy;
Ph, phenyl;
iPr, isopropyl;
XBP1, X-box binding protein-1;
BiP, Binding immunoglobulin Protein; and
WO95E, 4′-((5-((3-hydroxybenzyl)carbamoyl)-2,3-dimethyl-1H-indol-1-yl)methyl)-[1,1′-biphenyl]-2-carboxylic acid.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, diluents, and adjuvents which may improve solubility, deliverability, dispersion, stability, and/or conformational integrity of the compounds or conjugates thereof.

The term “active agent” as used herein refers to compounds as described herein or active conjugates thereof. A conjugate is a compound comprising an active agent covalently linked, directly or indirectly via a linker molecule, to a secondary compound, such as an antibody or fragment thereof. The active agent may be associated with a targeting moiety or molecule which is able to bind to a target cell or a portion of a target cell. The targeting moiety may be linked directly or indirectly to the active agent, or to the pharmaceutically acceptable carrier, vehicle, or diluent which contains or is associated with the active agent. The targeting moiety may be any molecule that can bind to another molecule. For example, a targeting moiety may include an antibody or its antigen-binding fragments, a receptor molecule, a chimeric antibody molecule, or an affinity reagent. As used herein, the term “targeting moiety” refers to a structure that binds or associates with a biological moiety or fragment thereof. As noted, in some embodiments, the targeting moiety may be an antibody. In some embodiments, the targeting moiety may be a monoclonal antibody (mAb). In some embodiments, the targeting moiety may be an antibody fragment, surrogate, or variant. In some embodiments, the targeting moiety may be a protein ligand. In some embodiments, the targeting moiety may be a protein scaffold. In some embodiments, the targeting moiety may be a peptide. In some embodiments, the targeting moiety may be RNA or DNA. In some embodiments, the targeting moiety may be a RNA or DNA fragment. In some embodiments, the targeting moiety may be a small molecule ligand.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the active agent to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. In addition, the active agent of the present disclosure may be designed to provide delayed, controlled, extended, and/or sustained release using formulation techniques which are well known in the art.

The term “topical” is used herein to define a mode of administration through an internal or external epithelial surface, such as but not limited to, a material that is administered by being applied externally to the eye or a nasal mucosa. A non-limiting example of topical administration is through the use of eyedrops or through the use of a nasally-administered aerosol.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of the active agent which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one”.

As utilized in accordance with the methods, compounds, and compositions of the present disclosure, the following terms, unless otherwise indicated, shall be understood to have the following meanings.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

Where used herein, the pronoun “we” is intended to refer to all persons involved in a particular aspect of the investigation disclosed herein and as such may include non-inventor laboratory assistants and collaborators working under the supervision of the inventor.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc., all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CAB ABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the active agent or composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

By “biologically active” is meant the ability of the active agent to modify the physiological system of an organism without reference to how the active agent has its physiological effects

Effectiveness of a method or use, such as a treatment that provides a potential therapeutic benefit or improvement of a condition or disease, can be ascertained by various methods and testing assays.

Use of the word “we,” “us,” and/or “our” as a pronoun in the present disclosure refers generally to laboratory personnel, technicians, or other contributors who assisted in laboratory procedures and data collection and is not intended to represent an inventorship role by said laboratory personnel, technicians, or other contributors in any subject matter disclosed herein.

Certain of the disclosed compounds may exist in various stereoisomeric forms. Stereoisomers are compounds that differ only in their spatial arrangement. Enantiomers are pairs of stereoisomers that are non-superimposable mirror images of one another, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms.

Compounds of the present disclosure may contain one or more asymmetrically-substituted carbon or nitrogen atoms and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³ C and ¹⁴ C.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, Selection and Use, 2^{nd} Revised Edition (2011), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### Chemical Group Definitions

When used in the context of a chemical group: “hydrogen” is —H; “deuterium” is -D; “hydroxy” means —OH; “borane” is —B; “oxo” is ═O; “carbonyl” is —C═O—; “carboxy” is —C(═O)OH (also written as —COOH or —CO₂H); the term “halogen” includes fluoro (fluorine, F), chloro (chlorine, Cl), bromo (bromine, Br), and iodo (iodine, I), “halo” means independently —F, —Cl, —Br or —I; “amino” is —NH₂; “hydroxyamino” is —NHOH; “nitro” is —NO₂; imino is ═NH; “cyano” is —CN; “isocyanate” is —N═C═O; “azido” is —N₃; in a monovalent context “phosphate” is —OP(O)(OH)₂ or a deprotonated form thereof; in a divalent context “phosphate” is —OP(O)(OH)O —or a deprotonated form thereof; “mercapto” is —SH; “thio” is ═S; “sulfonyl” is —SO₂—; and “sulfinyl” is —S(O)—.

In the context of chemical formulas, the symbol “—” means a single bond, “═” means a double bond, and “≡”means triple bond. The symbol “ [symbol] ” represents an optional bond, which if present is either single or double. The symbold “ [symbol] ” represents a single bond or a double bond. Thus, the formula

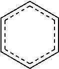

covers, for example,

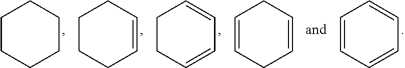

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

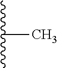

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

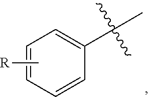

then the variable may replace any hydrogen atom attached to any of the ring atoms, including a depicted, implied, or expressly defined hydrogen, so long as a stable structure is formed.

When a variable is depicted as a “floating group” on a fused ring system, as for example the group “R” in the formula:

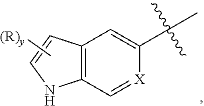

then the variable may replace any hydrogen attached to any of the ring atoms of either of the fused rings unless specified otherwise.

Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “Cₙ” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “C₅ olefin”, “C₅-olefin”, “olefin_{(C5)}”, and “olefin_{C5}” are all synonymous. When any of the chemical groups or compound classes defined herein is modified by the term “substituted”, any carbon atom(s) in the moiety replacing a hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic π system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. Where used herein alkyls, alkoxyls, haloalkyls, and haloalkoxyls are generally intended to refer to molecules having hydrocarbon chains that comprise 1, 2, 3, 4, 5, 6, 7, 8, 9, 10, 11, or 12 carbons, unless otherwise designated. The hydrocarbon chains may be straight or branched. Examples of alkyls include but are not limited to methyl, ethyl, propyl, isopropyl, and butyl. Alkoxy denotes an alkyl group which is linked to an oxygen atom. Examples of alkoxyls include but are not limited to methoxyl, ethoxyl, propoxyl, isopropoxyl, and butoxyl. Haloalkyls and haloalkoxyls are alkyls and alkoxyls which comprise at least one halogen atom such as chlorine, fluorine, bromine, or iodine.

The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The term “alkyl” includes straight or branched hydrocarbon groups having 1-10 carbon atoms and includes, for example, methyl, ethyl, n-propyl, isopropyl, n-butyl, sec-butyl, isobutyl, tert.-butyl, n-pentyl, n-hexyl, n-heptyl, n-octyl, n-nonyl, n-decyl, fluoromethyl, fluorochloromethyl, and trifluoromethyl, and the like. Alkyl groups may be optionally substituted with one or more substituents, such as halogens. The term “branched” should be understood to represent a linear straight chain hydrocarbon group having one or more lower alkyl groups such as methyl, ethyl or propyl, attached to it. The groups —CH₂— (methylene), —CH₂CH₂—, —CH₂C(CH₃)₂CH₂—, and —CH₂CH₂CH₂—are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂.

An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH_{3,}—NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e., —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

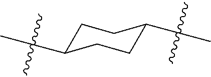 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” refers to an alkyl group containing at least one carbon-carbon double bond. Alkenyl groups may be optionally substituted with one or more substituents. The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure.

The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” refers to an alkyl group containing at least one carbon-carbon triple bond. Alkynyl groups may be optionally substituted with one or more substituents. The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl or cycloalkyl groups (carbon number limitation permitting) attached to the first aromatic ring or any additional aromatic ring present. If a cycloalkyl groups is present, such a group may be fused to one or more of the aromatic ring present. Non-limiting examples of aryl groups include phenyl (Ph), benzyl, methylphenyl, (dimethyl)phenyl, —C₆H₄CH₂CH₃ (ethylphenyl), naphthyl, and a monovalent group derived from biphenyl (e.g., 4-phenylphenyl).

The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

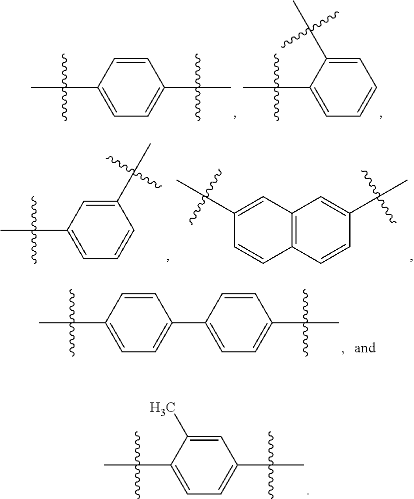

An “arene” refers to the class of compounds having the formula H—R, wherein R is aryl as that term is defined above. Benzene and toluene are non-limiting examples of arenes (aryl groups). When the term substituted aryl or substituted arene is used, one or more hydrogen atoms of the aryl or arene group has been independently replaced by —OH, —F, —Cl, —Br, —I, —NH₂, —NO₂, —CO₂H, —CO₂CH₃, —CN, —SH, —OCH₃, —OCH₂CH₃, alkoxy, —C(O)CH₃, —NHCH_{3,} —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group —alkanediyl—aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, the aromatic ring structures being one, two, three, or four ring structures each containing from three to nine ring atoms, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl.

The term “heteroaryl” includes aromatic mono- or bicyclic rings incorporating one or more (e.g., 1-4) heteroatoms selected from N, O, and S. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused 5- and 6-membered rings or two fused 6-membered rings. Each ring may contain up to about four heteroatoms typically selected from N, O, and S. Typically the heteroaryl ring will contain up to 3 heteroatoms, more usually up to 2, for example a single heteroatom. In one embodiment, the heteroaryl ring contains at least one ring N atom. The N atoms in the heteroaryl rings can be basic, as in the case of an imidazole or pyridine, or essentially non-basic as in the case of an indole or pyrrole nitrogen. In general, the number of basic N atoms present in the heteroaryl group, including any amino group substituents of the ring, will be less than 5. Examples of heteroaryl include, but are not limited to, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d] pyridazinyl, imidazo[2,1-b] thiazolyl, and i midazo[1,2-b][1,2,4]triazinyl.

Examples of heteroaryl groups comprising at least one N in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other rings is a non-aromatic, saturated or partially saturated ring, provided at least one ring contains one or more heteroatoms selected from N, O, and S. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, and 3,4-dihydro-2H-pyrido[3,2-b][1,4]oxazinyl.

The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroarenediyl does not preclude the presence of one or more alkyl or aryl groups (carbon number limitation permitting) attached to the aromatic ring or aromatic ring system. Non-limiting examples of heteroarenediyl groups include:

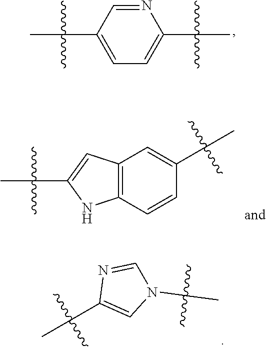 [SMILES: *c1ccc(*)nc1 ; *c1ccc2[nH]c(*)cc2c1 ; *c1cn(*)cn1]

The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH_{3,} —OCH₂CH₃, —C(O)CH₃, —NHCH₃, —NHCH₂CH₃, —N(CH₃)₂, —C(O)NH₂, —C(O)NHCH_{3,} —C(O)N(CH₃)₂, —OC(O)CH₃, —NHC(O)CH₃, —S(O)₂OH, or —S(O)₂NH_{2.}

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, the non-aromatic ring structures being one, two, three, or four ring structures each containing from three to nine ring atoms, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic.

Non-limiting examples of heterocycle (i.e., heterocycloalkyl) groups include cyclic ethers such as oxiranyl, oxetanyl, tetrahydrofuranyl, dioxanyl, and substituted cyclic ethers. Heterocycloalkyl rings comprising at least one N in a ring position include, for example, aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, tetrahydrotriazinyl, tetrahydropyrazolyl, tetrahydropyridinyl, homopiperidinyl, homopiperazinyl, 3,8-diaza-bicyclo[3.2.1]octanyl, 8-aza-bicyclo[3.2.1] octanyl, 2,5-Diaza-bicyclo[2.2.1]heptanyl and the like. Typical sulfur containing heterocycloalkyl rings include tetrahydrothienyl, dihydro-1,3-dithiol, tetrahydro-2H-thiopyran, and hexahydrothiepine. Other heterocycloalkyl rings include oxiranyl, oxetanyl, dihydrooxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolanyl, octahydrobenzofuranyl, octahydrobenzimidazolyl, and octahydrobenzothiazolyl.

In particular embodiments, the term heterocycle includes pyrrole, pyridine, imidazole, indole, skatole, methylindole, piperazine, piperidine, and pyrazine.

For heterocycles containing S, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. 1 and 2 oxo (═O) heterocyclyl groups include for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from N, O, or S, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the term “piperidine” or “morpholine” refers to a piperidin-1-yl or morpholin-4-yl ring that is linked via the ring nitrogen.

Cycloalkyls and aryls include, but are not limited to phenyl, toluene, cyclohexane, cyclopentane, piperidine, piperazine. Substituted cycloalkyls and aryls include, but are not limited to, aniline, benzoic acid, methyl-cyclohexane, and alkyl piperidines.

In particular embodiments the terms aryl and substituted aryl includes phenyl and benzyl, respectively.

Non-limiting examples of heterocycloalkyl groups include the term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings are connected with a covalent bond. As used herein, the term heterocycloalkanediyl does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

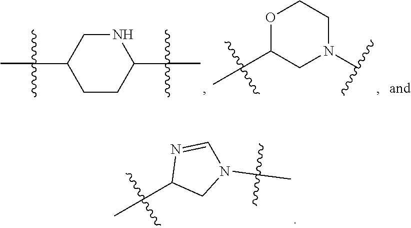 [SMILES: *C1CCC(*)NC1 ; *C1CN(*)CCO1 ; *C1CN(*)C=N1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively.

The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The term “hydroxypropyl” refers to three-carbon groups comprising one hydroxyl group and includes, but is not limited to, 2-hydroxypropyl and 1-hydroxypropan-2-yl. The term “dihydroxypropyl” refers to three-carbon groups comprising two hydroxyl groups and includes, but is not limited to, 1,3-dihydroxypropan-2-yl and 2,3-dihydroxypropyl.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms ortho, meta and para substitution are well understood in the art. For the absence of doubt, “ortho” substitution is a substitution pattern where adjacent carbons possess a substituent. “Meta” substitution is a substitution pattern where two substituents are on carbons one carbon removed from each other, i.e., with a single carbon atom between the substituted carbons. In other words there is a substituent on the second atom away from the atom with another substituent. “Para” substitution is a substitution pattern where two substituents are on carbons two carbons removed from each other, i.e., with two carbon atoms between the substituted carbons. That is, there is a substituent on the third atom away from the atom with another substituent.

Where used herein, the term “weak base” refers to compounds that accept protons weakly. Examples include but are not limited to ammonia and sodium bicarbonate. Where used herein, the term “weak acid” refers to compounds that have a weak tendency to donate protons. Examples include but are not limited to acetic acid, and citric acid. Where used herein, the term “strong base” refers to compounds that readily accepts protons, and the term “strong acid” refers to compounds that have a strong tendency to donate protons.

The active agents of the present disclosure may be present in the pharmaceutical compositions at any concentration that allows the pharmaceutical composition to function in accordance with the present disclosure; for example, but not by way of limitation, the active agents may be present in the composition in a range having a lower level selected from 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure.

Suitable carriers, vehicles, and other components that may be included in the formulation are described, for example, in Remington: The Science and Practice of Pharmacy, 21^{st} Ed. and 22^{nd} Ed. The term “pharmaceutically acceptable” means that the carrier is a non-toxic material that does not interfere with the effectiveness of the biological activity of the active agent. The characteristics of the carrier will depend on various factors, including but not limited to, the route of administration. For example, but not by way of limitation, the active agent may be dissolved in a physiologically acceptable pharmaceutical carrier or diluent and administered as either a solution or a suspension. Non-limiting examples of suitable pharmaceutically acceptable carriers include water, saline, dextrose solutions, fructose solutions, ethanol, or oils of animal, vegetative, or synthetic origin, or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the active agent and pharmaceutically acceptable carrier(s) (and other additional therapeutically active agent(s), if present). Examples of additional components that may be present include, but are not limited to, diluents, fillers, salts, buffers, preservatives, stabilizers, solubilizers, and other materials well known in the art. Another particular non-limiting example of an additional component that may be present in the pharmaceutical composition is a delivery agent, as discussed in further detail herein below.

Other embodiments of the pharmaceutical compositions of the present disclosure may include the incorporation or entrapment of the active agent in various types of drug delivery systems that function to provide targeted delivery, controlled release, and/or increased half-life to the active agent. For example, but not by way of limitation, it is possible to entrap the active agent in microcapsules prepared by coacervation techniques or by interfacial polymerization (for example, hydroxymethylcellulose or gelatin-microcapsules and poly-(methylmethacylate) microcapsules, respectively). It is also possible to entrap the active agent in macroemulsions or colloidal drug delivery systems (such as but not limited to, liposomes, albumin microspheres, microemulsions, nanoparticles, nanocapsules, and the like). Such techniques are well known to persons having ordinary skill in the art, and thus no further description thereof is deemed necessary.

In one particular, non-limiting example, the pharmaceutical composition may include a liposome in which the active agent is disposed. In addition to other pharmaceutically acceptable carrier(s), the liposome may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference.

In other non-limiting examples, the active agent of the present disclosure may be incorporated into particles of one or more polymeric materials, as this type of incorporation can be useful in controlling the duration of action of the active agent by allowing for controlled release from the preparations, thus increasing the half-life thereof. Non-limiting examples of polymeric materials that may be utilized in this manner include polyesters, polyamides, polyamino acids, hydrogels, poly(lactic acid), ethylene vinylacetate copolymers, copolymer micelles of, for example, PEG and poly(1-aspartamide), and combinations thereof.

The pharmaceutical compositions described or otherwise contemplated herein may further comprise at least one delivery agent, such as a targeting moiety, that assists in delivery of the active agent to a desired site of delivery, such as a pancreatic beta cell or liver cell.

The compositions of the present disclosure may be formulated for administration by any other method known or otherwise contemplated in the art, as long as the route of administration allows for delivery of the active agent so that the compounds can function in accordance with the present disclosure, e.g., to reduce ER stress. Examples of other routes of administration include, but are not limited to, oral, topical, retrobulbar, subconjunctival, transdermal, parenteral, subcutaneous, intranasal, intramuscular, intraperitoneal, intravitreal, and intravenous routes, including both local and systemic application routes.

Another non-limiting embodiment of the present disclosure is directed to a kit that contain one or more of any of the pharmaceutical compositions described or otherwise contemplated herein. The kit may further contain a second agent as described herein above for use concurrently with the pharmaceutical composition(s). If the composition present in the kit is not provided in the form in which it is to be delivered, the kit may further contain a pharmaceutically acceptable carrier, vehicle, diluent, or other agent for mixing with the active agent for preparation of the pharmaceutical composition. The kit including the composition and/or other reagents may also be packaged with instructions packaged for administration and/or dosing of the compositions contained in the kit. The instructions may be fixed in any tangible medium, such as printed paper, or a computer-readable magnetic or optical medium, or instructions to reference a remote computer data source such as a worldwide web page accessible via the internet.

The kit may contain single or multiple doses of the pharmaceutical composition which contains the active agent. When multiple doses are present, the doses may be disposed in bulk within a single container, or the multiple doses may be disposed individually within the kit; that is, the pharmaceutical compositions may be present in the kit in unit dosage forms to facilitate accurate dosing. The term “unit dosage forms” as used herein refers to physically discrete units suitable as unitary dosages for human subjects and other mammals; each unit contains a predetermined quantity of the active agent calculated to produce the desired therapeutic effect, in association with a suitable pharmaceutical excipient. Typical unit dosage forms of liquid compositions include prefilled, premeasured ampules or syringes; for solid compositions, typical unit dosage forms include pills, tablets, capsules, or the like. In such compositions, the active agent may sometimes be a minor component (from about 0.1 to about 50% by weight, such as but not limited to, from about 1 to about 40% by weight) with the remainder being various vehicles or carriers and processing aids helpful for forming the desired dosing form.

The active agent may be provided as a “pharmaceutically acceptable salt,” which refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297 (incorporated by reference herein in its entirety).

The amount of the active agent that is effective in the treatment described herein can be determined by the attending diagnostician, as one of ordinary skill in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diseases or other conditions involved; the degree, involvement, and/or severity of the diseases or conditions; the response of the individual subject; the particular active agent administered; the mode of administration; the bioavailability characteristics of the preparation administered; the dose regimen selected; the use of concomitant medication; and other relevant circumstances. A therapeutically effective amount of an active agent of the present disclosure also refers to an amount of the active agent which is effective in controlling, reducing, or ameliorating the condition to be treated.

Practice of the method of the present disclosure may include administering to a subject a therapeutically effective amount of the pharmaceutical composition (containing the active agent in any suitable systemic and/or local formulation, in an amount effective to deliver the dosages listed above. The dosage can be administered, for example, but not by way of limitation, on a one-time basis, or administered at multiple times (for example, but not by way of limitation, from one to five times per day, or once or twice per week). The pharmaceutical composition may be administered either alone or in combination with other therapies, in accordance with the inventive concepts disclosed herein.

Compositions of the active agent can be administered in a single dose treatment or in multiple dose treatments on a schedule and over a time period appropriate to the age, weight and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, or whether the composition is used for prophylactic or curative purposes. For example, in certain embodiments, the composition is administered once per month, twice per month, three times per month, every other week, once per week, twice per week, three times per week, four times per week, five times per week, six times per week, every other day, daily, twice a day, or three times a day. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) or vehicle to form a pharmacological composition. Pharmaceutically acceptable carriers can contain a physiologically acceptable compound that acts to, e.g., stabilize, or increase or decrease the absorption or clearance rates of the pharmaceutical compositions. Physiologically acceptable carriers and vehicles can include, for example, carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds, carriers, and vehicles include wetting agents, emulsifying agents, dispersing agents or preservatives.

When administered orally, the present compositions may be protected from digestion. This can be accomplished either by complexing the active agent with a composition to render it resistant to acidic and enzymatic hydrolysis or by packaging active agent in an appropriately resistant carrier such as a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical, transdermal administration, the agents are formulated into ointments, creams, salves, powders and gels. Transdermal delivery systems can also include, e.g., patches. The present compositions can also be administered in sustained delivery or sustained release mechanisms. For example, biodegradeable microspheres or capsules or other biodegradeable polymer configurations capable of sustained delivery of the active agent can be included herein.

For inhalation, the active agent can be delivered using any system known in the art, including dry powder aerosols, liquids delivery systems, air jet nebulizers, propellant systems, and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

The active agent can be delivered alone or as pharmaceutical compositions by any means known in the art, e.g., systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

In one aspect, the pharmaceutical formulations comprising the active agent are incorporated in lipid monolayers or bilayers, e.g., liposomes, such as shown in U.S. Pat. Nos. 6,110,490; 6,096,716; 5,283,185; and 5,279,833. Liposomes and liposomal formulations can be prepared according to standard methods and are also well known in the art, such as U.S. Pat. Nos. 4,235,871; 4,501,728 and 4,837,028.

In one aspect, the active agent is prepared with one or more carriers that will protect the active agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The active agent in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one example of a pharmaceutically suitable excipient.

Examples of routes of administration of the active agents described herein include parenteral injection, e.g., by subcutaneous, intramuscular or transdermal delivery. Other forms of parenteral administration include intravenous, intraarterial, intralymphatic, intrathecal, intraocular, intracerebral, or intracavitary injection. In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hanks' solution. Nonaqueous excipients such as fixed oils and ethyl oleate may also be used. An alternative excipient is 5% dextrose in saline. The excipient may contain minor amounts of additives such as substances that enhance isotonicity and chemical stability, including buffers and preservatives.

Formulated compositions comprising the active agent can be used for subcutaneous, intramuscular or transdermal administration. Compositions can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The active agents may be administered in solution. The formulation thereof may be in a solution having a suitable pharmaceutically acceptable buffer such as phosphate, Tris (hydroxymethyl) aminomethane-HCl or citrate, and the like. Buffer concentrations should be in the range of 1 to 100 mM. The formulated solution may also contain a salt, such as sodium chloride or potassium chloride in a concentration of 50 to 150 mM. An effective amount of a stabilizing agent such as mannitol, trehalose, sorbitol, glycerol, albumin, a globulin, a detergent, a gelatin, a protamine or a salt of protamine may also be included.

For example, but not by way of limitation, the therapeutically effective amount of an active agent used in the present disclosure will generally contain sufficient active agent to deliver in a range of from about 0.01 μg/kg to about 10 mg/kg (weight of active agent/body weight of patient). For example, but not by way of limitation, the composition will deliver about 0.1 μg/kg to about 5 mg/kg, and more particularly about 1m/kg to about 1 mg/kg.

Exemplary, non-limiting ranges for a therapeutically or prophylactically effective amount of the active agent include but are not limited to 0.001 mg/kg of the subject's body weight to 100 mg/kg of the subject's body weight, more typically 0.01 mg/kg to 100 mg/kg, 0.1 mg/kg to 50 mg/kg, 0.1 mg/kg to 40 mg/kg, 1 mg/kg to 30 mg/kg, or 1 mg/kg to 20 mg/kg, or 2 mg/kg to 30 mg/kg, 2 mg/kg to 20 mg/kg, 2 mg/kg to 15 mg/kg, 2 mg/kg to 12 mg/kg, or 2 mg/kg to 10 mg/kg, or 3 mg/kg to 30 mg/kg, 3 mg/kg to 20 mg/kg, 3 mg/kg to 15 mg/kg, 3 mg/kg to 12 mg/kg, or 3 mg/kg to 10 mg/kg, or 5 mg to 1500 mg, as a fixed dosage.

The composition is formulated to contain an effective amount of the active agent, wherein the amount depends on the animal to be treated and the condition to be treated. In certain embodiments, the active agent is administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

The dosage of an administered active agent for humans will vary depending upon such factors as the patient's age, weight, height, sex, general medical condition and previous medical history. In certain non-limiting embodiments, the recipient is provided with a dosage of the active agent that is in the range of from about 1 mg to 1000 mg as a single infusion or single or multiple injections, although a lower or higher dosage also may be administered. The dosage may be in the range of from about 25 mg to 100 mg of the active agent per square meter (m²) of body surface area for a typical adult, although a lower or higher dosage also may be administered. Examples of dosages that may be administered to a human subject further include, for example, 1 to 500 mg, 1 to 70 mg, or 1 to 20 mg, although higher or lower doses may be used. Dosages may be repeated as needed, for example, once per week for 4-10 weeks, or once per week for 8 weeks, or once per week for 4 weeks. It may also be given less frequently, such as every other week for several months, or more frequently, such as twice weekly or by continuous infusion.

Where used herein alkyls, alkoxyls, haloalkyls, and haloalkoxyls are generally intended to refer to molecules having hydrocarbon chains that comprise 1, 2, 3, 4, 5, 6, 7, 8, 9, 10, 11, or 12 carbons, unless otherwise designated. The hydrocarbon chains may be straight or branched. Examples of alkyls include but are not limited to methyl, ethyl, propyl, isopropyl, and butyl. Alkoxy denotes an alkyl group which is linked to an oxygen atom. Examples of alkoxyls include but are not limited to methoxyl, ethoxyl, propoxyl, isopropoxyl, and butoxyl. Haloalkyls and haloalkoxyls are alkyls and alkoxyls which comprise at least one halogen atom such as chlorine, fluorine, bromine, or iodine.

In at least certain embodiments, the present disclosure includes compounds having chemical structure (I) and includes methods of their use in treating disorders and conditions related to, for example, insulin resistance, obesity, diabetes type 2, NAFLD, and PPARγ S273 phosphorylation (or other related disorders or conditions described elsewhere herein), and for promoting conversion of white adipocytes to beige and/or brown adipocytes. Chemical structure (I) is:

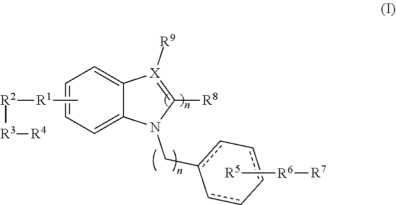

wherein
n₁=1 or 2;
n₂=1 or 2;

### X═C or N;

R¹ is selected from the group consisting of CH₂, C═O, and NH;
R² is selected from the group consisting of CH₂, C═O, NH;
R³ is selected from the group consisting of alkyl, aryl, substituted aryl, and heterocycle;
R⁴ is one or independently two of the group consisting of H, OH, alkyl, alkoxy, OCH₃, NH₂, SO₂, CF₃, COOH, SO₂NH₂, CN, CONH₂, phenyl, substituted phenyls, benzyl, and heterocycles;
R⁵ is selected from the group consisting of alkyl, cycloalkyl, aryl, and heterocycle;
R⁶ is selected from the group consisting of alkyl, cycloalkyl, aryl, CH₂, NH, O, OCH₃, SH, SCH₃;
R⁷ is selected from the group consisting of H, COOH, NH, SO₂NH, B(OH)₂, CF₃, and heterocycle;
R⁸ is selected from the group consisting of H, D, alkyl, cycloalkyl, aryl, substituted aryl, and heterocycle; and
R⁹ is selected from the group consisting of H, D, alkyl, cycloalkyl, aryl, substituted aryl, and heterocycle.

In at least one embodiment of a compound with chemical structure (I), at least one of or both of R⁸ and R⁹≠H or D. In certain embodiments, R³ is selected from phenyl, and benzyl. In certain embodiments, R⁶ is selected from phenyl, and benzyl. In certain embodiments, R⁴ comprises a single group selected from H, OH, alkyl, alkoxy, OCH₃, NH₂, SO₂, CF₃, COOH, SO₂NH₂, CN, CONH₂, phenyl, substituted phenyls, benzyl, and heterocycles. In certain embodiments, when R³ is a phenyl or benzyl, R⁴ comprises a pair of groups selected independently from H, OH, alkyl, alkoxy, OCH₃, NH₂, SO₂, CF₃, COOH, SO₂NH₂, CN, CONH₂, phenyl, substituted phenyl, benzyl, and heterocycles.

In at least certain embodiments, the present disclosure includes compounds having chemical structure (II) and includes methods of their use in treating disorders and conditions related to, for example, insulin resistance, obesity, diabetes type 2, NAFLD, and PPARγ S273 phosphorylation (or other related disorders or conditions described elsewhere herein), and for promoting conversion of white adipocytes to beige and/or brown adipocytes. Chemical structure (II) is:

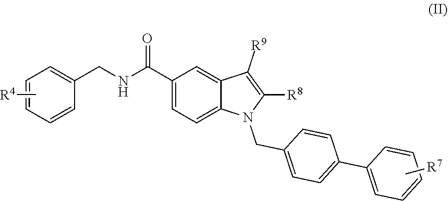

wherein
R⁴ is one or independently two of the group consisting of H, OH, alkyl, alkoxy, OCH₃, NH₂, SO₂, COOH, SO₂NH₂, CN, CONH₂, phenyl, substituted phenyl, and heterocycle;
R⁷ is selected from the group consisting of H, COOH, NH, SO₂NH, B(OH)₂, CF₃, and heterocycle;
R⁸ is selected from the group consisting of H, D, alkyl, cycloalkyl, aryl, substituted aryl, and heterocycle; and
R⁹ is selected from the group consisting of H, D, alkyl, cycloalkyl, aryl, substituted aryl, and heterocycle.

In at least one embodiment of a compound with chemical structure (II), at least one of or both of R⁸ and R⁹≠H. In certain embodiments, R⁴ comprises a single group selected from H, OH, alkyl, alkoxy, OCH₃, NH₂, SO₂, CF₃, COOH, SO₂NH₂, CN, CONH₂, phenyl, substituted phenyls, benzyl, and heterocycles. In certain embodiments, R⁴ comprises a pair of groups selected independently from H, OH, alkyl, alkoxy, OCH₃, NH₂, SO₂, CF₃, COOH, SO₂NH₂, CN, CONH₂, phenyl, substituted phenyls, benzyl, and heterocycles.

In one embodiment of the present disclosure, the compound has the chemical formula 4′-((5-((3 -hydroxybenzyl)carb amoyl)-2,3-dimethyl-1H-indol-1-yl)methyl)-[1,1′-biphenyl]-2-carboxylic acid, and is designated herein as WO₉₅E. In at least certain embodiments WO₉₅E can be used to treat disorders and conditions related to, for example, insulin resistance, obesity, diabetes type 2, NAFLD, and PPARγ S273 phosphorylation (or other related disorders or conditions described elsewhere herein), and for promoting conversion of white adipocytes to beige and/or brown adipocytes. WO₉₅E and has the chemical structure:

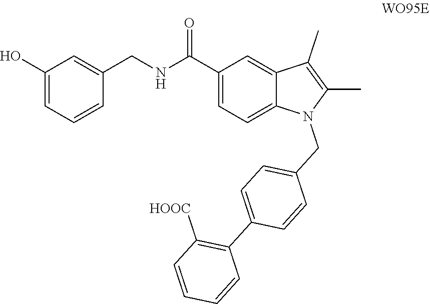 [SMILES: Cc1c(C)n(Cc2ccc(-c3ccccc3C(=O)O)cc2)c2ccc(C(=O)NCc3cccc(O)c3)cc12]

In other non-limiting embodiments of the present disclosure, the following compounds are included:

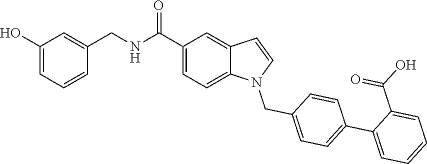 [SMILES: O=C(NCc1cccc(O)c1)c1ccc2c(ccn2Cc2ccc(-c3ccccc3C(=O)O)cc2)c1]

4′-((5-((3-hydroxybenzyl)carbamoyl)-1H-indol-1-yl)methyl)-[1,1′-biphenyl]-2-carboxylic acid

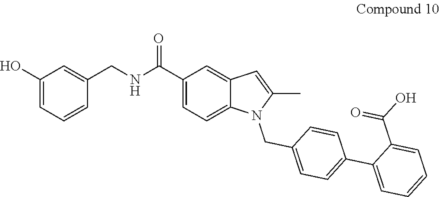 [SMILES: Cc1cc2cc(C(=O)NCc3cccc(O)c3)ccc2n1Cc1ccc(-c2ccccc2C(=O)O)cc1]

4′-((5-((3-hydroxybenzyl)carbamoyl)-2-methyl-1H-indol-1-yl)methyl)-[1,1′-biphenyl]-2-carboxylic acid

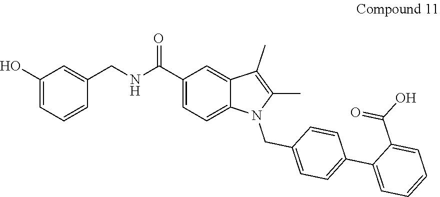 [SMILES: Cc1c(C)n(Cc2ccc(-c3ccccc3C(=O)O)cc2)c2ccc(C(=O)NCc3cccc(O)c3)cc12]

4′-((5-((3-hydroxybenzyl)carbamoyl)-2,3-dimethyl-1H-indol-1-yl)methyl)-[1,1′-biphenyl]-2-carboxylic acid

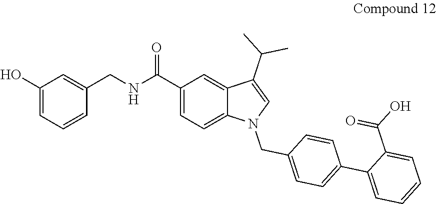 [SMILES: CC(C)c1cn(Cc2ccc(-c3ccccc3C(=O)O)cc2)c2ccc(C(=O)NCc3cccc(O)c3)cc12]

4′-((5-((3-hydroxybenzyl)carbamoyl)-3-isopropyl-1H-indol-1-yl)methyl)-[1,1′-biphenyl]-2-carboxylic acid

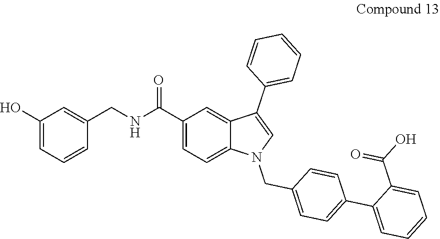 [SMILES: O=C(NCc1cccc(O)c1)c1ccc2c(c1)c(-c1ccccc1)cn2Cc1ccc(-c2ccccc2C(=O)O)cc1]

4′4(54(3-hydroxybenzyl)carbamoyl)-3-phenyl-1H-indol-1-yl)methyl)-[1,1′-biphenyl]-2-carboxylic acid

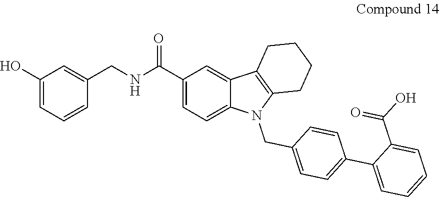 [SMILES: O=C(NCc1cccc(O)c1)c1ccc2c(c1)c1c(n2Cc2ccc(-c3ccccc3C(=O)O)cc2)CCCC1]

4′-((6-((3-hydroxybenzyl)carbamoyl)-1,2,3,4-tetrahydro-9H-carbazol-9-yl)methyl)-[1,1′-biphenyl]-2-carboxylic acid

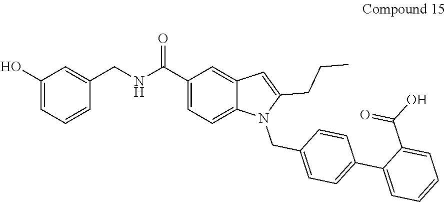 [SMILES: CCCc1cc2cc(C(=O)NCc3cccc(O)c3)ccc2n1Cc1ccc(-c2ccccc2C(=O)O)cc1]

4′-((5-((3-hydroxybenzyl)carbamoyl)-2-propyl-1H-indol-1-yl)methyl)-[1,1′-biphenyl]-2-carboxylic acid

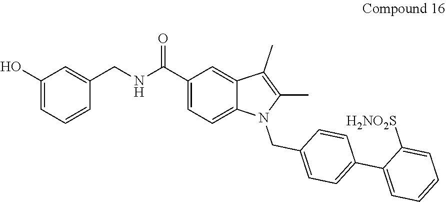 [SMILES: Cc1c(C)n(Cc2ccc(-c3ccccc3S(N)(=O)=O)cc2)c2ccc(C(=O)NCc3cccc(O)c3)cc12]

N-(3-hydroxybenzyl)-2,3-dimethyl-1-((2′-sulfamoyl-[1,1′-biphenyl]-4-yl)methyl)-1H-indole-5-carboxamide

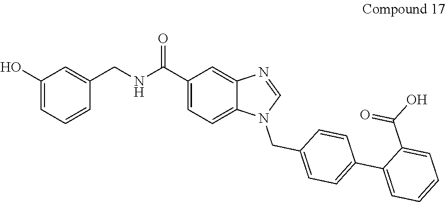 [SMILES: O=C(NCc1cccc(O)c1)c1ccc2c(c1)ncn2Cc1ccc(-c2ccccc2C(=O)O)cc1]

4′-((5-((3-hydroxybenzyl)carbamoyl)-1H-benzo[d]imidazol-1-yl)methyl)-[1,1′-biphenyl]-2-carboxylic acid

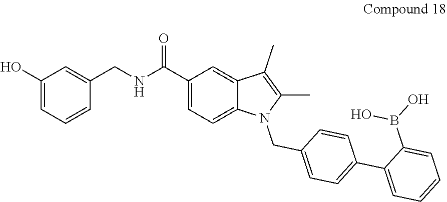 [SMILES: Cc1c(C)n(Cc2ccc(-c3ccccc3B(O)O)cc2)c2ccc(C(=O)NCc3cccc(O)c3)cc12]

(4′-((5-((3-hydroxybenzyl)carbamoyl)-2,3-dimethyl-1H-indol-1-yl)methyl)-[1,1′-biphenyl]-2-yl)boronic acid

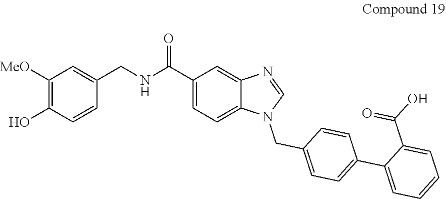 [SMILES: COc1cc(CNC(=O)c2ccc3c(c2)ncn3Cc2ccc(-c3ccccc3C(=O)O)cc2)ccc1O]

4′-((5-((4-hydroxy-3-methoxybenzyl)carbamoyl)-1H-benzo[d]imidazol-1-yl)methyl)-[1,1′-biphenyl]-2-carboxylic acid

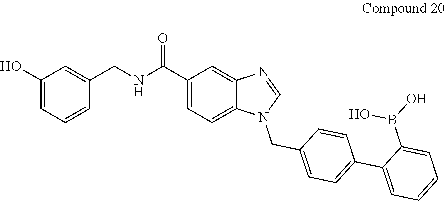 [SMILES: O=C(NCc1cccc(O)c1)c1ccc2c(c1)ncn2Cc1ccc(-c2ccccc2B(O)O)cc1]

(4′-((5-((3-hydroxybenzyl)carbamoyl)-1H-benzo[d]imidazol-1-yl)methyl)-[1,1′-biphenyl]-2-yl)boronic acid

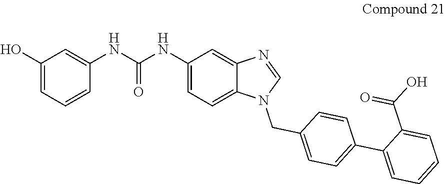 [SMILES: O=C(Nc1cccc(O)c1)Nc1ccc2c(c1)ncn2Cc1ccc(-c2ccccc2C(=O)O)cc1]

4′-((5-(3-(3-hydroxyphenyl)ureido)-1H-benzo[d]imidazol-1-yl)methyl)-[1,1′-biphenyl]-2-carboxylic acid

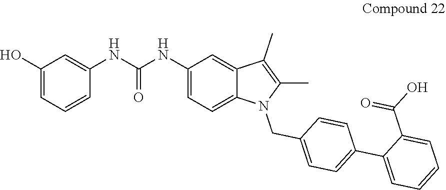 [SMILES: Cc1c(C)n(Cc2ccc(-c3ccccc3C(=O)O)cc2)c2ccc(NC(=O)Nc3cccc(O)c3)cc12]

4′-((5-(3-(3-hydroxyphenyl)ureido)-2,3-dimethyl-1H-indol-1-yl)methyl)-[1,1′-biphenyl]-2-carboxylic acid

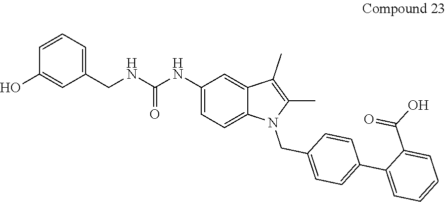 [SMILES: Cc1c(C)n(Cc2ccc(-c3ccccc3C(=O)O)cc2)c2ccc(NC(=O)NCc3cccc(O)c3)cc12]

4′-((5-(3-(3-hydroxybenzyl)ureido)-2,3-dimethyl-1H-indol-1-yl)methyl)-[1,1′-biphenyl]-2-carboxylic acid

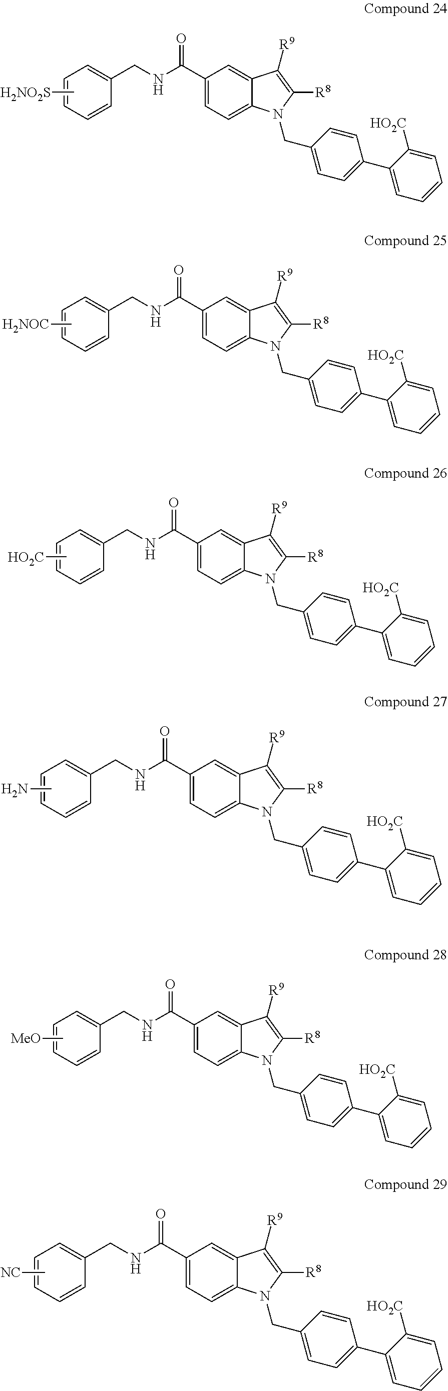

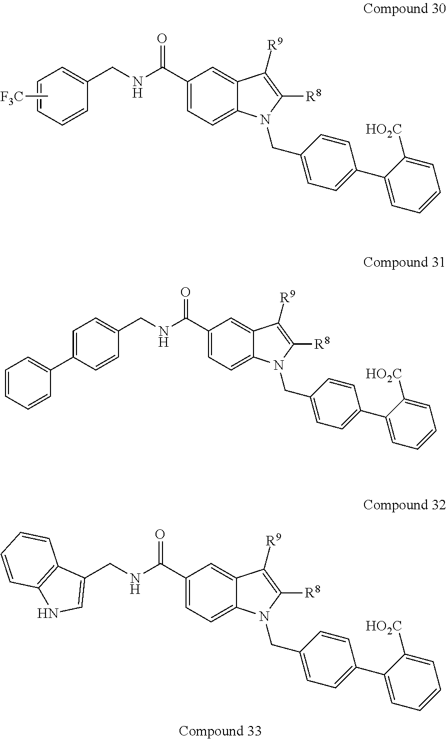

### EXAMPLES

Certain novel embodiments of the present disclosure, having now been generally described, will be more readily understood by reference to the following examples, which are included merely for purposes of illustration of certain aspects and embodiments of the present disclosure, and are not intended to be limiting. The following detailed examples are to be construed, as noted above, only as illustrative, and not as limiting of the present disclosure in any way whatsoever. Those skilled in the art will promptly recognize appropriate variations from the various compositions, structures, components, procedures, and methods.

### MATERIALS AND METHODS

### Chemicals, Cell Culture Reagents, and Plasmids

The 3T3-L1 mouse fibroblasts and HEK-293 (ATCC, Manassas, VA, USA) were maintained at 37° C. in a 5% CO₂, humidified atmosphere. The cells were cultured in Dulbecco's modified Eagle's medium (DMEM) containing 4.5 g/liter glucose with 10% fetal bovine serum (FBS; HyClone, Logan, UT, USA). PolarScreen TM PPARγ-Competitor Assay Kit was purchased from Thermo Fisher Scientific (Waltham, US). Bright-Glo™ Luciferase Assay System was purchased from Promega (Madison, US). Oil Red O was purchased from Alfa Aesar (Haverhill, MA, USA). Rosiglitazone was purchased from Sigma. UHC1 and WO₉₅E were synthesized in-house. DMSO was used as solvent and as vehicle control at 0.1% in cell-based assays. Plasmids pBabe bleo human PPARγ2 (#11439) and PPRE X3-TK-luc (#1015) were acquired from Addgene.

### In Vitro Competition Binding Assay

The in vitro competition binding assays was performed using PolarScreen™ PPARγ-Competitor Assay Kit according to the manufacturer's instructions. Briefly, compounds at 2-fold titration serial concentrations were incubated with GST-tagged PPARγ ligand-binding domain (LBD, 34 nM) and Fluormone PPAR Green tracer (9 nM) for 4 hours in the dark at room temperature in a black 384-well assay plate (Corning Glass cat. no. 677). The florescence polarization value (mP) was measured at 485 nm (excitation) and at 535 nm (emission) using a microplate reader.

### In Silico Docking Simulations

The docking simulation was performed using the AutoDock Vina program. The crystal structure of PPARγ LBD (PDB ID: 5GTO) was used for the docking simulation and subsequent structural analysis. The grid dimensions for PPARγ LBD protein was 44×44×44 grid points with spacing 0.375 Å between the grid points and centered on the ligand for protein (−27.376, 18.813, 2.589 coordinates). A compound was docked in the binding pocket site using the highest accuracy mode of docking.

### PPARγ Transactivation Reporter Assay

HEK-293 cells were seeded overnight, co-transfected with plasmids expressing human PPARγ2 and 3×PPRE-luc (both from Addgene) followed by incubation for 16 h, and then re-seeded in a 384-well plate and incubated for 24 h. Compounds were added and then incubated another 24 h. Luciferase activity was analyzed in each well using Bright-Glo Luciferase kit (Promega).

### Adipocyte Differentiation

3T3-L1 preadipocytes were differentiated. In brief, cells at around 60%-70% confluence were induced for differentiation with DMEM containing 10% FBS, 1 μg/ml insulin, 1 μM dexamethasone, and 0.5 mM IBMX for 48 h in the presence or absence of compounds and were then exposed in maintenance medium (1 μg/mL insulin in 10% FBS/DMEM) containing compounds. The medium was renewed every 2 days for total 8 days of differentiation when the cells became differentiated and lipid droplets were apparent. For primary preadipocyte differentiation, isolation of primary preadipocytes was performed. Briefly, fat depots were digested in PBS containing collagenase D (1.5 U/ml) at 37° C. for 40-45 min. The primary cells were filtered through 70 μm cell strainer and centrifuge at 700 g to collect stromal vascular fraction (SVF). The SVF cell pellets were plated on collagen coated plates. Adipocyte differentiation was induced by treating confluent cells with DMEM containing 10% FBS, 1 μg/m1 insulin, 1 μM dexamethasone, and 0.5 mM IBMX. Two days later, the medium was changed to maintenance medium as described above for 3T3-L1 cells. The cells were cultured in the presence or absence of WO₉₅E.

Oil Red O staining

Differentiated adipocytes were rinsed with phosphate-buffered saline (PBS) and then fixed with 4% formaldehyde for 30 min. After removal of the formaldehyde, the cells were washed with PBS three times. Subsequently, 0.3% Oil Red O staining solution was added to each well for 1 h incubation at 37° C. followed by washing with PBS three times. Staining of the lipid droplets were photographed using an inverted microscope (ECLIPSE TS100-F; Nikon).

### Animal Study

C57BL/6J male mice were obtained from Jackson laboratory (Bar Harbor, ME) and maintained on a 12 h light (6:00 AM to 6:00 PM)—12 h dark (6:00 PM to 6:00 AM) cycle at an ambient temperature of 22±1° C. All animals had access to diet and water ad libitum. All procedures involving animals were approved by the Institutional Animal Care and Use Committee of the University of Oklahoma Health Science Center. All experiments were performed with age-matched male mice. The mice were fed a high-fat diet (HFD) (60% kcal fat, Bio-Serv, NJ, USA) starting from the age of 6 weeks until study end. At the 8-week HFD, mice were randomly divided into two groups and were administered daily with either vehicle (10% DMSO, 5% tween-20) or WO₉₅E (15 mg/kg of body weight) for 4 weeks through the route of intraperitoneal injection. Food intake was determined manually by measuring the amount of input food subtracting the amount of the food remaining in the food hopper and the crumbs on the bedding of the cage each day for three days during the 4^{th} week of the treatment. Packed cell volume (PCV) was measured with blood drawn from tail on a LW Scientific E8 centrifuge (LW Scientific, Lawrenceville, Georgia, USA) microhematocrit centrifuge at the end of study. For all other measurements, the blood was drawn at the time of euthanization.

### Glucose Tolerance Test and Insulin Tolerance Test

Intraperitoneal glucose tolerance test (ipGTT) was performed after 6-h fasting. Blood glucose levels were measured at 0, 15, 30, 60, and 120 min glucometer (OneTouch Ultra 2 Meter) after an intraperitoneal administration of glucose at dose of 1.5 g/kg body weight. Intraperitoneal insulin tolerance test (ipITT) was performed after 6 h fasting. Blood glucose levels were measured at 0, 15, 30, 60, and 120 min after an intraperitoneal administration of human insulin at dose of 1.2 IU/kg body weight.

### Biochemical Analysis

Serum insulin (ALPCO, NH, USA), serum cholesterol TG (Cayman Chem., MI, USA) and FFAs (Bioassay system, CA, USA) were determined by ELISA.

### Body Composition Assessment

Body lean and fat composition was determined by EchoMRI test.

### Indirect Calorimetry Measurements

The mice were individually housed in chambers of a Promethion Core Monitoring system (Sable Systems, Las Vegas, NV, USA). After a 1-day acclimation period, their oxygen consumption and carbon dioxide production were measured for the next three consecutive days. The respiratory exchange ratio and energy expenditure were calculated using standard equations

### Histology and Immunohistochemistry

Liver, heart, bone, adipose tissues were dissected and immediately fixed in 4% paraformaldehyde (Sigma-Aldrich), paraffin embedded, and stained with hematoxylin and eosin. For Immunofluorescence staining, the eWAT and iWAT paraffin sections were incubated with primary antibody (rabbit polyclonal to F4/80 [1:500, Cat #: 70076], Cell Signaling Technology) overnight at 4° C. after deparaffinization. The slides were washed thrice with PBS with 0.2% Triton and then incubated in secondary antibody, Alexa 488 anti-rabbit (1:500, Jackson ImmunoResearch, PA) for 1.5 h at room temperature. Tissue sections were imaged on the Olympus Fluoview 1000 laser-scanning confocal microscope (Center Valley, PA).

RNA Extraction and qRT-PCR

Total RNA (2 μg) was isolated from tissues or cells using TRIzol reagents (Life Technologies) and reverse transcribed using oligo d(T) primers (New England Biosystems) and SuperScript IV reverse transcription kit (Applied Biosystems). qPCR was performed in a CFX96 Touch Real-Time PCR detection system using SYBR Green mix (Applied Biosystems). The amplification program was as follows: initial denaturation at 95° C. for 15 min, followed by 40 cycles of 95° C. for 15 s, 60° C. for 1 min, and 40° C. for 30 s. Relative mRNA expression was determined by the ΔΔCt method normalized to TBP mRNA. The sequences of primers used in this study were shown in Table 1 in U.S. Provisional Application Ser. No. 63/419,049, the entirety of which is incorporated herein by reference.

### Western Blotting

Proteins were extracted with RIPA buffer containing protease and phosphatase inhibitors (Thermo) and then centrifuged for 10 min at 10,000 g. A 30 μg sample of protein was separated on a 10% SDS polyacrylamide gel and transferred to polyvinylidene-fluoride membrane for 1 h at 4° C. at 40 V. The membrane was blocked in TBS (10 mm Tris-HCl, pH 7.4, 150 mm NaCl) containing 5% non-fat dry milk for 1 h at room temperature and was probed with primary antibodies followed by the appropriate HRP-conjugated secondary antibodies (Anti-rabbit IgG, #7074, or Anti-mouse IgG, #7076, 1:5000 Cell Signaling Technology). The primary antibodies used were: anti-Ser-273 PPARγ (bs-4888R, Bioss Antibodies), anti-PPARγ antibody (sc-271392, Santa Cruz Biotechnology), anti-UCP1 (14670S, Cell Signaling Technology, Beverly, MA, USA).

### Synthesis of WO₉₅E

The steps for synthesizing the compound WO₉₅E are shown in Synthetic Scheme I.

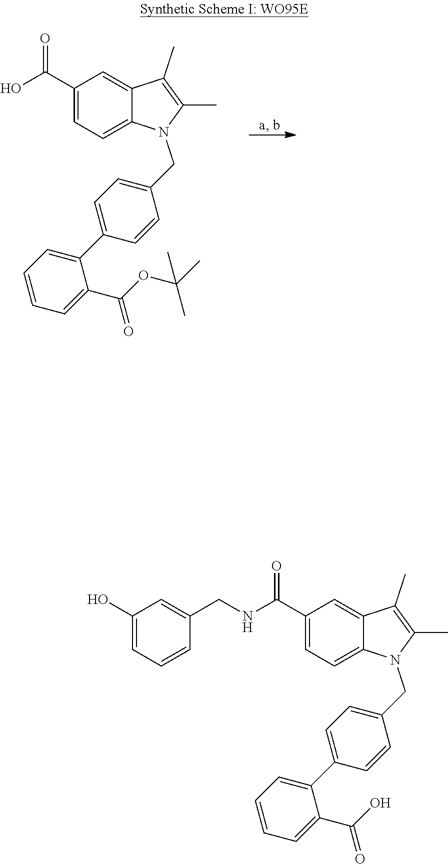 [SMILES: Cc1c(C)n(Cc2ccc(-c3ccccc3C(=O)OC(C)(C)C)cc2)c2ccc(C(=O)O)cc12 ; Cc1c(C)n(Cc2ccc(-c3ccccc3C(=O)O)cc2)c2ccc(C(=O)NCc3cccc(O)c3)cc12]

In Synthetic Scheme I the reagents and conditions in step (a) were HATU, DIPEA, and DCM at rt, and in step (b) were TFA/DCM at ambient temperature (described in more detail below). Reagents and solvents were obtained from commercial suppliers and were used without further purification. Reactions using air- or moisture sensitive reagents were performed under an atmosphere of Argon or Nitrogen. Reactions were monitored by TLC. Flash chromatography was performed with 230-400 mesh silica gel, NMR spectra were measured on Bruker 400 MHz spectrometers. Chemical shifts are reported in ppm in the indicated solvent with TMS as an internal standard. Data are reported in the form: chemical shift (multiplicity, coupling constants, and integration). Multiplicities are recorded by the following abbreviations: s, singlet; d, doublet; t, triplet; q, quartet; m, multiplet; br, broad. Analytical HPLC and ESI-MS analyses were performed using either Agilent or Krats MS 80 mass spectrometer. Tested compounds were evaluated on the Agilent HPLC systems and determined to be ≥95% pure.

### General Procedure: Amino Acid Coupling

To a mixture of the 1-(2′-(tert-butoxycarbonyl)-[1,1′-biphenyl]-4-yl)methyl)-2,3-dimethyl-1H-indole-5-carboxylic acid (1 equivalents) in DCM was added DIEA (2.5 equivalents) and HATU (1 equivalents). The mixture was stirred for 5 min, and then the 3-(aminomethyl)phenol (1 equivalents) was added. The reaction mixture was stirred at room temperature for 30 min. The completion of the reaction was monitored by TLC. The solvent was removed in vacuo to obtain the crude which was purified by flash chromatography to provide tert-butyl 4′-((5-((3-hydroxybenzyl)carbamoyl)-2,3-dimethyl-1H-indol-1-yl)methyl)-[1,1′-biphenyl]-2-carboxylate.

### Deprotection

TFA was added to solution of tert-butyl 4′-((5-((3-hydroxybenzyl)carbamoyl)-2,3-dimethyl-1H-indol-1-yl)methyl)-[1,1′-biphenyl]-2-carboxylate in DCM at room temperature. The reaction was stirred at room temperature for 30 min. The completion of the reaction was monitored by TLC. The solvent was removed to obtain the crude which was purified by Flash chromatography was performed with 230-400 mesh silica gel to obtain 4′-((5-((3-hydroxybenzyl)carbamoyl)-2,3-dimethyl-1H-indol-1-yl)methyl)-[1,1′-biphenyl]-2-carboxylic acid (WO₉₅E).

4′-((5-((3-hydroxybenzyl)carbamoyl)-2,3-dimethyl-1H-indol-1-yl)methyl)-[1,1′-biphenyl]-2-carboxylic acid (WO₉₅E)

1 H NMR (DMSO-d₆, 400 MHz) δ: 9.27 (s, 1H), 8.81 (t, J =5.7 Hz, 1H), 8.11 (s, 1H), 7.66 (m, 2H), 7.45 (m, 3H), 7.31 (d, J=7.5 Hz, 1H), 7.24 (d, J=7.4 Hz, 2H), 7.09 (t, J=7.8 Hz, 1H), 6.99 (d, J=7.6 Hz, 2H), 6.74 (m, 2H), 6.60 (d, J=7.9 Hz, 1H), 5.46 (s, 2H), 4.42 (d, J=5.6 Hz, 2H), 2.31 (s, 3H), 2.26 (s, 3H). ¹³C NMR (100MHz, DMSO-d6) δ: 168.5, 166.0, 156.2, 140.5, 139.3, 138.6, 136.5, 136.1, 132.9, 129.5, 129.2, 128.0, 127.8, 127.4, 124.8, 123.9, 119.0, 116.6, 116.5, 112.8, 112.4, 107.6, 106.1, 44.4, 8.9, 7.6.

### Statistical Analysis

All data are presented as the mean±SEM of the indicated number of replicates. Data were analyzed using the unpaired two-tailed Student's t-test and p<0.05 was considered statistically significant. For energy expenditure experiments, the data was analyzed for group effect (ANOVA) along with the mass and interaction effects as necessary (generalized linear model, GLM).

### RESULTS

### Identification and Characterization of a Novel PPARγ Partial Agonist WO₉₅E

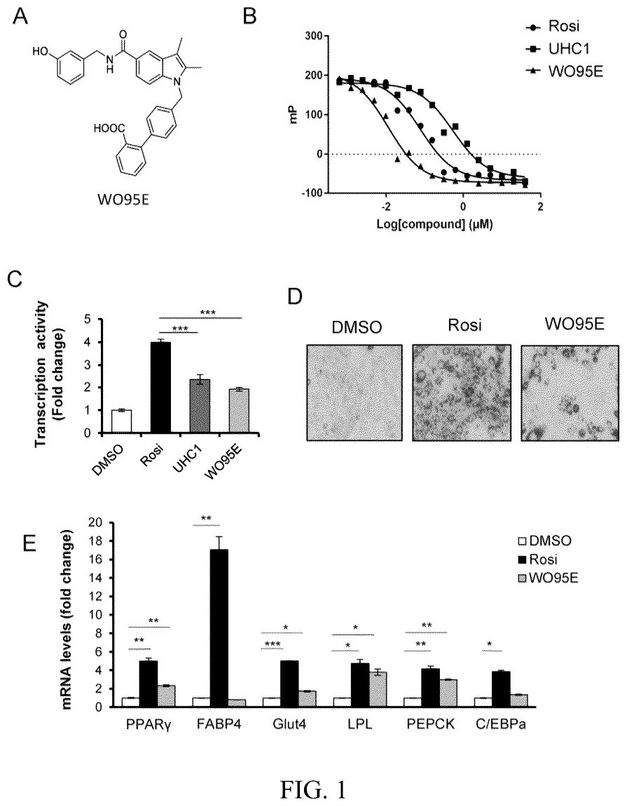

Indole derivatives such as SR1664 and UHC1 have been identified as synthetic ligands of PPARγ (Choi J H, Banks A S, Kamenecka T M, Busby S A, Chalmers M J, Kumar N, et al. Antidiabetic actions of a non-agonist PPARγ ligand blocking Cdk5-mediated phosphorylation. Nature. 2011;477(7365):477-81; Choi S S, Kim E S, Koh M, Lee S J, Lim D, Yang Y R, et al. A novel non-agonist peroxisome proliferator-activated receptor gamma (PPARγ) ligand UHC1 blocks PPARγ phosphorylation by cyclin-dependent kinase 5 (CDK5) and improves insulin sensitivity. The Journal of biological chemistry. 2014;289(38):26618-29). Although both compounds exhibited anti-diabetic activity without causing side effects associated with TZD use, they suffered from poor PPARγ binding affinity and pharmacokinetic properties. We have since synthesized a series of indole-based analogs, including the analog WO₉₅E shown herein (FIG. 1(A)), as PPARγ ligands. We assessed the binding affinity of WO₉₅E to PPARγ by performing an in vitro competition binding assay using purified PPARγ ligand binding domain (LBD). We found that WO₉₅E directly binds to PPARγ in a dose-dependent manner with a half-maximum inhibitory concentration (IC₅₀) of ˜11 nmol/l (FIG. 1(B)), which is approximately 70-fold lower than that of UHC1, indicative of WO₉₅E being a potent synthetic ligand of PPARγ.

We next determined the effect of WO₉₅E on the transactivation activity of PPARγ. PPARγ acts as a transcription factor to activate the expression of its target genes by binding to the PPAR-responsive element (PRE) in their promoters. To determine the effect of WO₉₅E on PPARγ transactivation activity, we transfected HEK293 cells with plasmids expressing PPARγ and 3XPRE-Luciferase reporter in the presence of WO₉₅E. As shown in FIG. 1(C), compared to the full agonist TZD drug Rosi which markedly activated the reporter, WO₉₅E increased the reporter activity only weakly, at ˜15% and 33% of potency as Rosi and UHC1, respectively. We noted that although it was originally reported as a non-agonist PPARγ ligand, UHC1 activated the reporter at approximately 45% of potency as Rosi. Together, these results indicate that WO₉₅E acts as a PPARγ partial agonist, displaying a much higher PPARγ binding affinity than indole derivatives SR1664 and UHC1 and minimal PPARγ transactivation activity.

We then investigated the effects of WO₉₅E on PPARγ function. As PPARγ is the master regulator of adipogenesis, we used a cell-based adipocyte differentiation assay to determine whether WO₉₅E affects this process. PPARγ activity is essential for 3T3-L1 mouse embryonic fibroblast cells to differentiate into adipocytes. When 3T3-L1 cells were incubated with the differentiation media, as expected, addition of Rosi markedly induced the differentiation of adipocytes, as indicated by the widespread presence of lipid droplets with positive Oil Red O staining (an indication of adipocyte formation) compared to little or no Oil Red O staining in the presence of DMSO control (FIG. 1(D)). Strikingly, WO₉₅E treatment only marginally increased Oil Red O staining level (FIG. 1(D)), indicative of its minimal effect on adipogenesis, thus corroborating the notion that WO₉₅E is a partial agonist of PPARγ. Next, we investigated the impact of WO₉₅E on the expression of genes responsible for adipogenesis. PPARγ regulates the expression level of various adipogenic genes. As expected, Rosi treatment of differentiated 3T3-L1 pre-adipocytes significantly upregulated the mRNA levels of adipogenic genes PPARγ, FABP4, Glut4, PEPCK, and C/EBPa (FIG. 1(E)). In contrast, in line with the weak effect of WO₉₅E on adipocyte differentiation, WO₉₅E only minimally or moderately increased the mRNA levels of these PPARγ dependent adipogenic genes (FIG. 1(E)).

### WO₉₅E Inhibits PPARγ Phosphorylation and Regulates PPARγ Phosphorylation-Dependent Genes

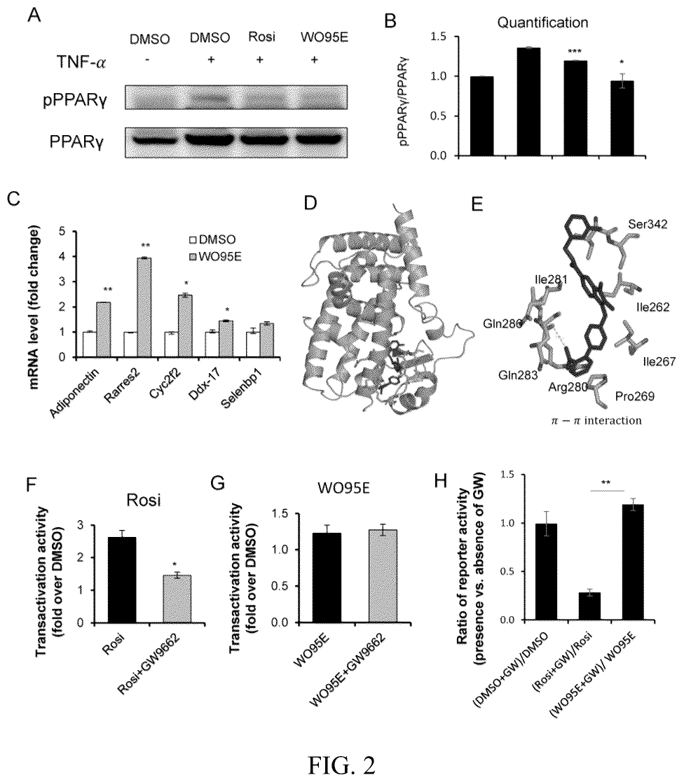

As noted, indole derivatives SR1664 and UHC1 were previously shown to inhibit PPARγ phosphorylation at 5273. As PPARγ de-phosphorylation at S273 is known to be critical for PPARγ-mediated insulin sensitivity, we therefore investigated whether WO₉₅E inhibits PPARγ phosphorylation at S273. Indeed, like Rosi and UHC1, WO₉₅E significantly inhibited the S273 phosphorylation of PPARγ in 3T3-L1 differentiated adipocytes, using a PPARγ phospho-S273-specific antibody (FIG. 2(A-B)). Next, as PPARγ S273 phosphorylation is associated with the repression of a set of genes that are believed to be responsible for insulin sensitivity, we examined the effect of WO₉₅E on the S273 phosphorylation-repressed genes. Our results showed that WO₉₅E treatment led to the upregulation of the mRNA levels of a number of the PPARγphosphorylation-dependent genes, including adiponectin, cycp2f2, Ddx-17, Rarres2, and Selenbp1 (FIG. 2(C)). Together, our data indicate that WO₉₅E inhibits PPARγ phosphorylation at S273 and up-regulates the PPARγ-phosphorylation-dependent genes.

Next, it was reported that PPARγ ligands such as SR1664 and UHC1 inhibit PPARγ S273 phosphorylation by binding to a noncanonical alternative binding pocket (ABP, which contains S273) of PPARγ, which blocks the PPARγ S273 phosphorylation from CDK5. We used an in silico docking modeling method (Autodock) to simulate WO₉₅E′ s mode of binding to PPARγ. Our simulation indicated that WO₉₅E does not bind to the canonical PPARγ ligand-binding pocket (LBP) containing H3, H3-4 loop, H11 and H12, which full agonist Rosi binds; instead, WO₉₅E binds to the ABP comprising H2′-H3, β-sheet, and the Ω loop (FIG. 2(D)). The model indicates that WO₉₅E binding shifts the position of S273 (FIG. 2(E)), which, without wishing to be bound by theory, could explain its suppression of S273 phosphorylation. To rule out the possibility that WO₉₅E binds to the canonical LBP of PPARγ, we employed a potent PPARγ antagonist GW9662 which binds and forms the irreversible covalent bond with the canonical PPARγ LBP (IC₅₀ 3.3 nM). As expected, GW9662 largely eliminated the ability of the full agonist Rosi to activate the 3×PPRE reporter (FIG. 2(F,H)), consistent with the prediction that Rosi is incapable of binding to the canonical PPARγ LBP in the presence of GW9662. In contrast, WO₉₅E retained its moderate ability to activate the reporter with GW9662 co-treatment (FIG. 2(G,H)), suggesting that WO₉₅E binds PPARγ via an alternative site.

### WO₉₅E Improves Glucose Tolerance and Insulin Sensitivity in Diet-Induced Obese Mice

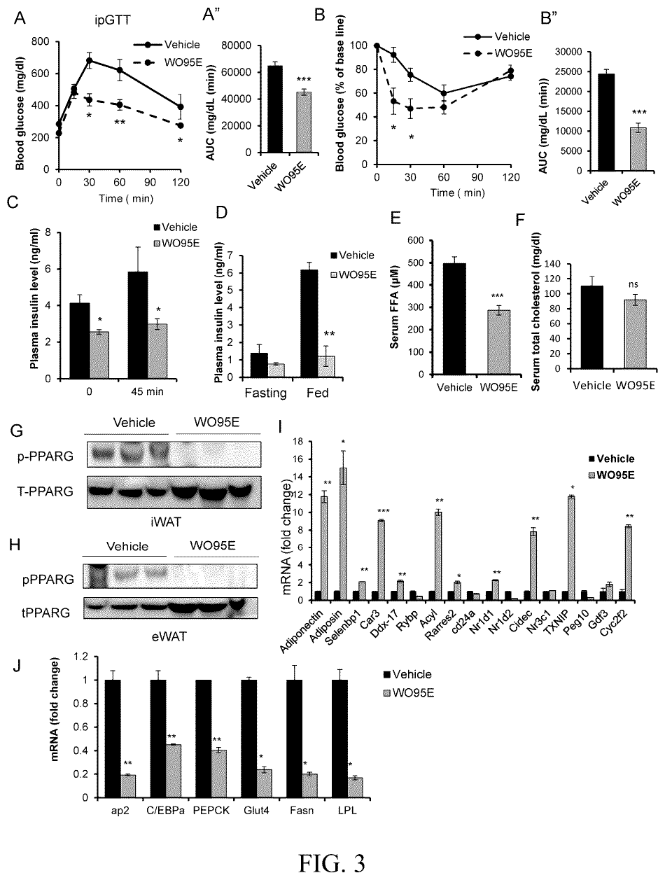

We next interrogated whether WO₉₅E improves insulin sensitivity in diet-induced obesity (DIO) mice with established insulin resistance. For this, we treated DIO mice with WO₉₅E or vehicle via intraperitoneal injection once daily for 4 weeks. We observed that WO₉₅E treatment significantly improved glucose tolerance with lower peak glucose level and decreased AUC (area under the curve) in response to a bolus of exogenous glucose compared to their vehicle-treated counterpart (FIG. 3(A, A′)). Insulin sensitivity was also enhanced in DIO mice treated with WO₉₅E (FIG. 3(B,B′)). Next, we found that DIO mice treated with WO₉₅E exhibited markedly decreased fasting and refeeding plasma levels of insulin (FIG. 3(C)), indicative of increased insulin sensitivity in the WO₉₅E-treated mice. Similar trend in insulin levels was observed in mice following an exogenous bolus of glucose (FIG. 3(D)). Collectively, these data indicate that WO₉₅E improves insulin sensitivity and glucose disposal in DIO mice. Similarly, we also observed that WO₉₅E decreased the serum level of free fatty acid (FIG. 3(E)), whereas total serum cholesterol showed downward trend but did not reach statistically significant (FIG. 3(F)) and triglyceride level was unchanged (data not shown).

We next investigated whether the improvement of insulin sensitivity in WO₉₅E-treated DIO mice correlates with its effect on PPARγ phosphorylation at S273 in adipose tissues. First, we assessed the phosphorylation status of adipose tissues from DIO mice treated with WO₉₅E or vehicle. As shown in FIG. 3(G), WO₉₅E treatment markedly inhibited PPARγ phosphorylation in inguinal white adipose tissue (iWAT), as indicated by probing with a PPARγ phospho-5273-specific antibody. Similar results were obtained in epididymal white adipose tissue (eWAT) (FIG. 3(H)). Next, we examined the effect of WO₉₅E on the expression of S273 phosphorylation-dependent genes. Our results showed that the mRNA levels of genes including Adiponectin, Adipsin, selenbp1, Car3, Ddx-17, Acyl, Rarres2, and, Cidec, which are known to be repressed by PPARγ phosphorylation, were up-regulated by WO₉₅E treatment (FIG. 3(I)), whereas genes involved in lipogenesis or adipogenesis were inhibited (FIG. 3(J)) in eWAT. Therefore, similar to that seen in cultured adipocytes, WO₉₅E suppresses PPARγ phosphorylation and up-regulates the PPARγ-phosphorylation-repressed genes in adipose tissue.

### WO₉₅E Protects Against Diet-Induced Obesity and Increases Energy Expenditure

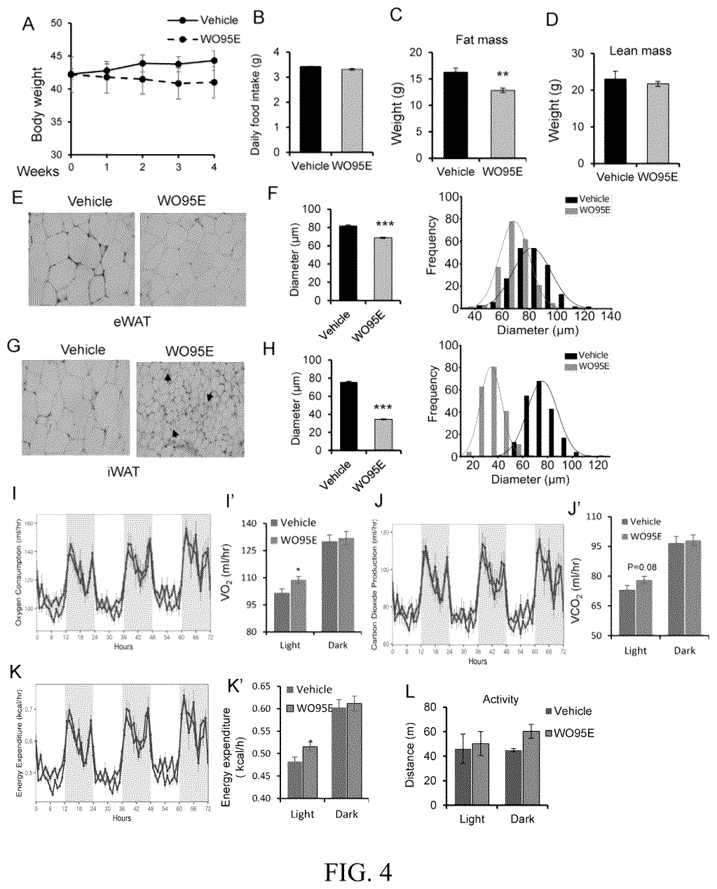

In addition to insulin sensitization, we also observed that WO₉₅E also protected against diet-induced obesity. As shown in FIG. 4(A), while vehicle-treated mice continue to increase body weight under high fat diet (HFD), WO₉₅E treatment reduced body weight during the period of 4-week treatment. This reduction was not due to the decrease in food intake as we detected the same amount of daily food intake in mice between WO₉₅E and vehicle treatments (FIG. 4(B)). Body composition analysis using EchoMRI further revealed a significant decrease in the fat mass in WO₉₅E-treated DIO mice (FIG. 4(C)) with no noticeable change in lean mass (FIG. 4(D)), suggesting that the body weight reduction is largely due to fat mass reduction. We next analyzed adipocytes in WAT sections and observed that adipocytes were significantly smaller with a lower average cell size in both eWAT (FIG. 4(E-F)) and iWAT (FIG. 4(G-H)) in WO₉₅E-treated DIO mice than in vehicle-treated mice.

The finding that WO₉₅E protects against HFD-induced obesity without altering food intake (FIG. 4(A-B)) suggests an increase in energy expenditure. We therefore investigated energy expenditure in DIO mice treated with WO₉₅E using indirect calorimetry. We observed that average oxygen consumption (V02) was higher in DIO mice treated with WO₉₅E than with vehicle (FIG. 4(1, I′)). We similarly detected that average carbon dioxide production (VCO₂) was increased in WO₉₅E-treated DIO mice but with a p value at 0.08 for daytime (FIG. 4(J,J′)). Accordingly, WO₉₅E treatment significantly heightened mean energy expenditure compared to vehicle treatment (FIG. 4(K,K′)). In contrast, WO₉₅E-treated DIO mice exhibited similar respiratory exchange ratio (RER) value as vehicle-treated DIO mice, suggesting that WO₉₅E does not alter the relative contribution of carbohydrate and lipids to elicit an increase in energy expenditure (data not shown). We also detected an upward trend in the overall activity of DIO mice treated with WO₉₅E, but with the difference being statistically insignificant (FIG. 4(L)).

### Conversion of White to Brown Adipose Tissue in WO₉₅E-Treated Mice

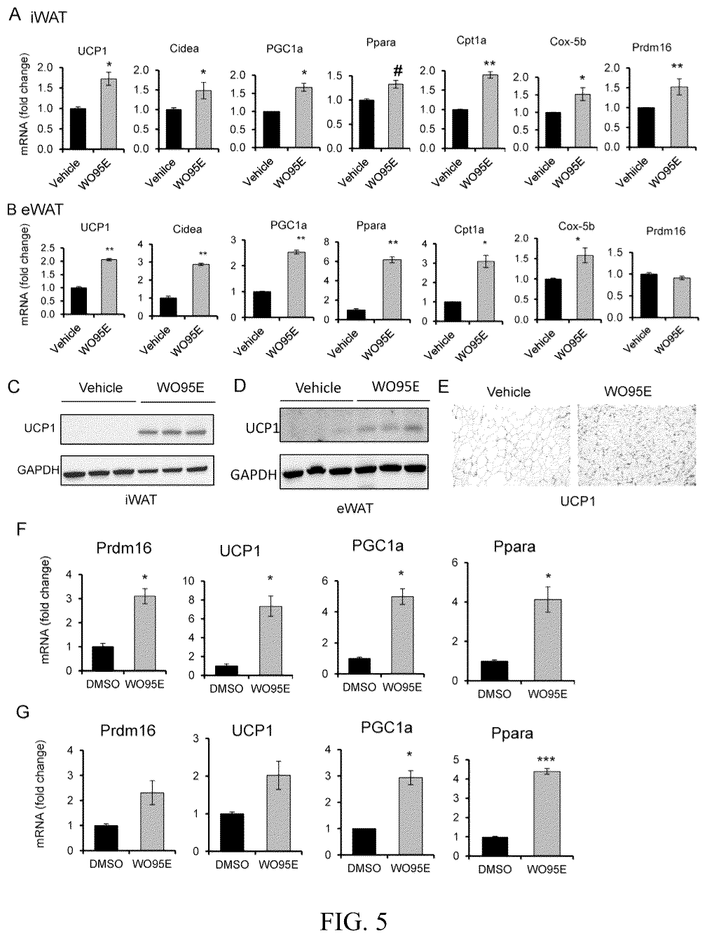

Brown adipose tissue (BAT) is specialized for energy expenditure. BAT activation and brown remodeling of WAT increases energy expenditure. We therefore investigated whether WO₉₅E increases overall energy expenditure by promoting the browning of WAT. We first examined the expression of genes that are involved in thermogenic and mitochondrial functions and enriched in BAT, including Prdm16, UCP1, Cidea, Ppara, CPT1a, Cox-5b, and PGC1α, in WAT. We detected significantly increased mRNA levels of Prdm16, Cidea, Ppara, CPT1a, UCP1, Cox5b, and PGC1α in the iWAT of DIO mice subjected to WO₉₅E treatment compared to vehicle (FIG. 5(A)). Similar results were obtained in the eWAT with the exception of Prdm16 mRNA (FIG. 5(B)). We also observed increased mRNA expression levels of most of these genes in BAT tissues (data not shown), suggesting the prevention/reversal of “whitening” of high fat diet-induced BAT. In addition, Western blotting revealed a dramatic induction of UCP1 protein, a brown adipocyte marker, in both iWAT and eWAT (FIG. 5(C-D)), and an increase in the level of UCP1 protein in BAT tissue (data not shown). Similarly, histochemical staining also showed that UCP1 was enhanced in the WAT from WO₉₅E-treated DIO mice (FIG. 5(E)). Furthermore, we frequently observed the occurrence of multilocular brown/beige adipocytes (multiple small lipids droplets, a hallmark of brown-like cells) in iWAT from DIO mice treated with WO₉₅E (FIG. 4(G)). Together, these results demonstrate that WO₉₅E induces conversion of white adipocytes to brown/beige adipocytes.

To investigate whether WO₉₅E achieves the browning effects by directly acting on adipose tissues, we treated primary preadipocytes freshly isolated from iWAT with WO₉₅E. WO₉₅E significantly increased the expression levels of Prdm16, UCP1, PCG1α, and Ppara, genes involved in thermogenic/brown activities, but not in the adipogenic genes, in the iWAT adipocytes (FIG. 5(F). Similar results were obtained in WO₉₅E-treated primary preadipocytes isolated from eWAT (FIG. 5(G)). These results show that WO₉₅E acts directly on adipose tissues for the white to brown conversion.

### WO₉₅E Ameliorates Inflammation in Adipose Tissue

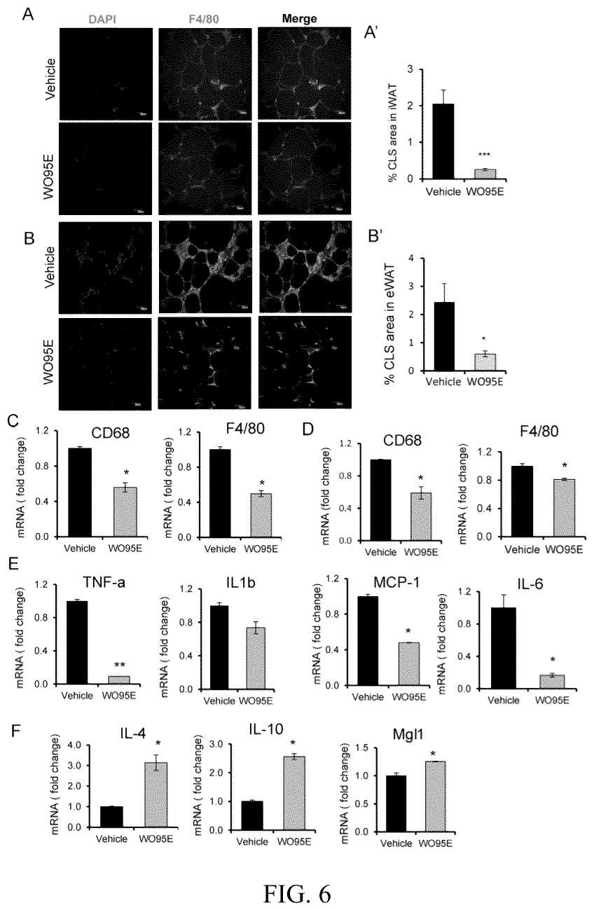

In obesity, there is significant accumulation of macrophages in the adipose tissue. It has been proposed that the polarized recruitment/accumulation of pro-inflammatory M1 macrophages from the anti-inflammatory alternatively activated macrophages (M2) is critical for the development of systemic insulin resistance. We interrogated whether WO₉₅E could impact the adipose tissue inflammation. First, we measured the area of crown-like structures (CLS) in the subcutaneous and visceral fat depots. CLSs are characteristic elements that are formed upon the infiltration and recruitment of macrophages (marked by F4/80) around dead adipocytes in the adipose tissues. As shown in FIG. 6(A-B′), the areas of CLS were dramatically reduced in both iWAT and eWAT of DIO mice treated with WO₉₅E relative to vehicle-treated mice. We next investigated whether WO₉₅E treatment affects the expression of genes involved in inflammation in the adipose tissue. We found that the mRNA levels of genes that encode pan-macrophage markers (CD68 and F4/80) were significantly decreased in the SVF portion of both inguinal (FIG. 6(C)) and epididymal (FIG. 6(D)) adipose tissues of DIO mice treated with WO₉₅E compared to vehicle. Likewise, WO₉₅E also down-regulated the mRNA levels of genes that encode proinflammatory mediators [TNFα, IL1b, IL-6, and monocyte chemoattractant protein-1 (mcp-1), which are all enriched in M1 macrophages] in iWAT (FIG. 6(E)) and in eWAT (data not shown). In contrast, the mRNA levels of genes that encode the markers (IL4, IL10, and Mg1I) for M2 macrophages were up-regulated or unchanged in the SVF portion of both inguinal and epididymal adipose tissues from WO₉₅E-treated DIO mice (FIG. 6(F). These results indicate that WO₉₅E lessens the adipose tissue inflammation in obesity.

### WO₉₅E Improves Hepatic Steatosis in DIO Mice

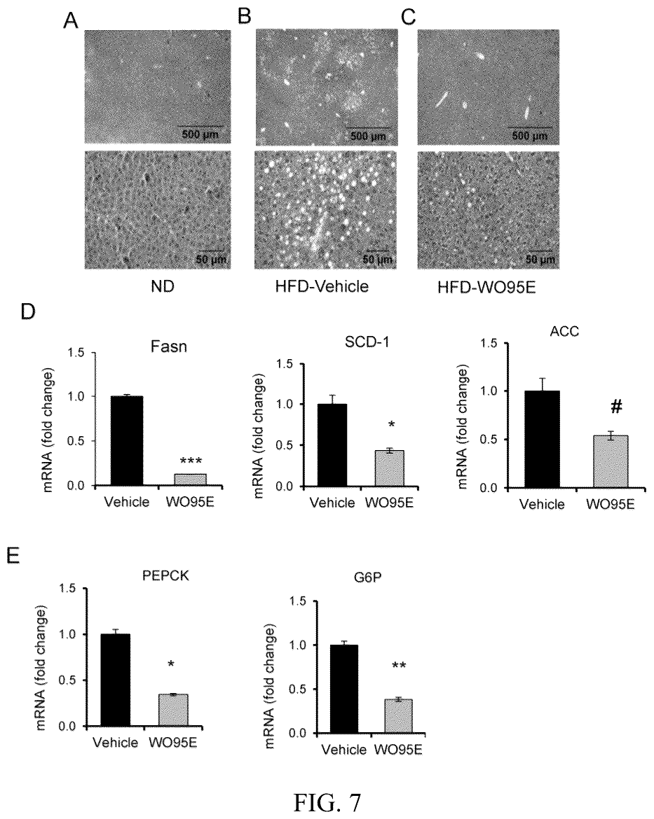

A common comorbidity of obesity and insulin resistance is NAFLD. Given that WO₉₅E improves systemic insulin in DIO mice and that PPARγ full agonist TZDs improve the condition of fatty liver, we investigated the potential effects of WO₉₅E on liver steatosis in obesity. First, we examined the liver histology in H&E-stained sections. We observed the presence of numerous lipid droplets in the liver, a hallmark of live steatosis, in the vehicle-treated DIO mice compared to that in the BL/6 mice (FIG. 7(A-B)). Strikingly, WO₉₅E treatment markedly reduced the number of the lipid droplets in the liver of DIO mice compared to vehicle treatment (FIG. 7(B-C)). We next examined the expression of genes involved in lipogenesis in the liver of the WO₉₅E- and vehicle-treated DIO mice and found that the lipogenic genes FASN, stearoly-coA desaturase-1 (SCD-1), and acetyl coA-carboxylase (ACC) were down-regulated in the liver of WO₉₅E-treated DIO mice compared to vehicle-treated mice (FIG. 7(D)). Furthermore, because NAFLD is associated with an impaired suppression of hepatic glucose output, we assessed the expression of genes involved in gluconeogenesis, including glucose-6-phosphatase catalytic subunit (G6PC) and PEPCK. We found that the expression levels of PEPCK and G6PC in the liver from WO₉₅E-treated DIO mice were also significantly decreased compared to that of vehicle-treated ones (FIG. 7(E)).

### WO₉₅E has Minimal TZD-Associated Side-Effects

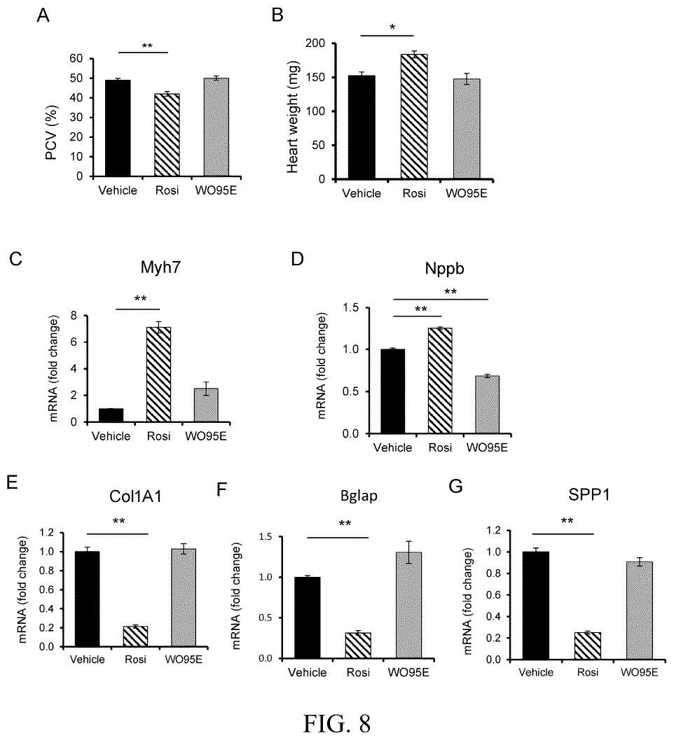

PPARγ full agonist TZDs have been linked to multiple adverse side effects. We investigated whether WO₉₅E, as a partial PPARγ ligand that also inhibits its phosphorylation, reduces TZD-linked adverse side effects. Fluid retention, adiposity and weight gain are among the most frequent side effects associated with TZD use. However, WO₉₅E did not cause weight gain and adiposity, instead it resulted in decreases in both body weight and fat mass (FIG. 4(A, C)). In addition, as shown in FIG. 8(A), while treatment with Rosi caused a significant reduction in packed-cell volume (PCV), which is indicative of hemodilution, the PCV was unchanged following WO₉₅E treatment.

TZD use is also associated with cardiac hypertrophy or dysfunction which are partially attributable to fluid retention and weight gain. We investigated the effect of WO₉₅E on the heart weight and found that unlike the TZD drug Rosi, which induces a significant increase in heart weight relative to vehicle, WO₉₅E had no effect on weight gain in the heart (FIG. 8(B)). We then assessed the expression of cardiac genes associated with heart failure or hypertrophy, such as myosin heavy chain β(β-Mhc, also Myh7) and natriuretic peptide B (Nppb). As expected, Rosi treatment increased the mRNA levels of Myh7 (FIG. 8(C)) and Nppb (FIG. 8(D)) compared to vehicle treatment, observations that are consistent with previous findings. In contrast, the mRNA levels of Myh7 and Nppb were not significantly altered or even lowered in the hearts of DIO mice treated for WO₉₅E (FIG. 8(C-D)). TZD is also associated with a decrease in bone formation and bone mineral density and hence presents an increased risk for bone fracture. As shown in FIG. 8(E-G), 4-week treatment of WO₉₅E in DIO mice had no apparent effect on the mRNA levels of genes involved in bone formation, including osteocalcin (Bglap), osteopontin (SPP1) and ColA1, whereas Rosi treatment decreased their expression significantly.

### DISCUSSION

Obesity-associated insulin resistance is a core feature of type 2 diabetes (T2D) and other metabolic disorders. PPARγ full agonist TZD drugs were once widely used to treat T2D due to their potent effect on insulin sensitization. However, the TZD drugs are associated with serious adverse effects, including weight gain, fluid retention, and congestive heart failure as full agonism is responsible for the activation of genes that are associated with these side effects. As a consequence, the prescription of TZD drugs has fallen tremendously recently. Research focus has now been shifted to understanding mechanisms that decouple the insulin sensitivity from TZD-associated side effects. Recent studies have shown that the insulin-sensitizing effect of TZD drugs is achieved through their inhibition of PPARγ pS273, which is independent of their classical full agonism. Indeed, several PPARγ ligands such as SR1664 and UHC1 have been shown to suppress PPARγ S273 phosphorylation and exert glucose-lowering activity, without causing the commonly observed side effects associated with TZDs. But chemicals that bind to PPARγ with high affinity are still lacking.

The present disclosure describes a class of indole derivatives that acts as PPARγ partial agonists with a high binding affinity (e.g., for WO₉₅E, IC₅₀˜11 nM), while blocking PPARγ S273 phosphorylation. The compounds improve glucose disposal and insulin resistance obese subjects. Blockage of PPARγ S273 phosphorylation apparently decouples TZD-associated side effects from insulin sensitivity. Several side effects such as weight gain, adiposity and fluid retention can occur within a short time after the administration of TZDs, but unlike the TZD drug Rosi which increases body weight, WO₉₅E, for example, does not cause weight gain; instead, it reduces body weight (FIG. 4(A)) and adiposity (FIG. 4(C, E-H). WO₉₅E also does not appear to cause Rosi-associated water retention, cardiac hypertrophy, or bone density-associated gene expression (FIG. 8). Together, these results demonstrate that WO₉₅E can improve insulin sensitization without the incurrence of adverse side effects.

Another significant effect of the present compounds is the promotion of white-to-brown adipocyte conversion, as seen by an increase in adipocytes with multilocular appearance in the WAT of WO₉₅E-treated mice (FIG. 4(G)). In line with the known role of brown adipocytes in consuming energy reserves through non-shivering thermogenesis, we observed that WO₉₅E treatment increases oxygen consumption, CO₂ production, and energy expenditure in DIO mice (FIG. 4(I-K)). Consistently, genes involved in mitochondrial thermogenesis are up-regulated in WAT as well as BAT in WO₉₅E-treated animals. Further experiments on isolated preadipocytes treated with WO₉₅E indicate that WO₉₅E acts directly on adipose tissues to promote the browning of WAT. PPARγ agonist TZD drugs have previously been shown to possess the ability to activate the brown remodeling of WAT. It has been further reported that only TZDs, but not non-TZD partial agonists tested, were capable of inducing the brown conversion of white adipocyte tissue, leading to the conclusion by some that the full PPARγ agonism is required for the browning. An alternative interpretation for these results is that the presence of TZD moiety in the tested full agonists may account for the positive effect on browning. The findings of the present disclosure are that the novel non-TZD partial agonist WO₉₅E of PPARγ promotes the browning of WAT, indicating that neither the TZD backbone nor full agonism is required for white-to-brown adipocyte conversion. Instead, WO₉₅ is thought to induce browning in the same fashion as it improves insulin resistance, through the inhibition of PPARγ S273 phosphorylation.

As demonstrated herein, the present disclosure describes novel indole-based PPARγpartial agonists that inhibit PPARγ phosphorylation and have potent insulin sensitizing effects without causing TZD-associated adverse effects. The compounds promote energy expenditure and protect against obesity by inducing the conversion of white fat to brown-like or beige fat as the first non-TZD partial agonist. The compounds can be used, therefore, for example, in therapies for reducing obesity and weight-gain, reducing insulin resistance, and treating obesity-associated liver steatosis, nonalcoholic fatty liver disease (NAFLD), and type 2 diabetes.

In certain embodiments, the present disclosure is directed to compound having the chemical structure (I):

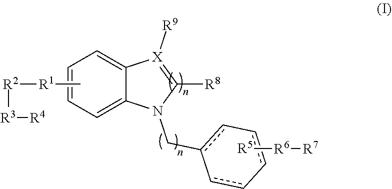

wherein, n₁=1 or 2; n₂=1 or 2; X═C or N; R¹ is selected from the group consisting of CH₂, C═O, and NH; R² is selected from the group consisting of CH₂, C═O, NH; R³ is selected from the group consisting of alkyl, aryl, substituted aryl, and heterocycle; R⁴ is one or independently two of the group consisting of H, OH, alkyl, alkoxy, OCH₃, NH₂, SO₂, CF₃, COOH, SO₂NH₂, CN, CONH₂, phenyl, substituted phenyls, benzyl, and heterocycles; R⁵ is selected from the group consisting of alkyl, cycloalkyl, aryl, and heterocycle; R⁶ is selected from the group consisting of alkyl, cycloalkyl, aryl, CH₂, NH, O, OCH₃, SH, SCH₃; R⁷ is selected from the group consisting of H, COOH, NH, SO₂NH, B(OH)₂, CF₃, and heterocycle; R⁸ is selected from the group consisting of H, D, alkyl, cycloalkyl, aryl, substituted aryl, and heterocycle; and R⁹ is selected from the group consisting of H, D, alkyl, cycloalkyl, aryl, substituted aryl, and heterocycle. In certain embodiments, at least one of or both of R⁸ and R⁹≠H or D. In certain embodiments R³ is selected from phenyl and benzyl. In certain embodiments R⁴ is a single group selected from H, OH, alkyl, alkoxy, OCH₃, NH₂, SO₂, CF₃, COOH, SO₂NH₂, CN, CONH₂, phenyl, substituted phenyls, benzyl, and heterocycles. In certain embodiments R⁴ is a pair of groups selected independently from H, OH, alkyl, alkoxy, OCH₃, NH₂, SO₂, CF₃, COOH, SO₂NH₂, CN, CONH₂, phenyl, substituted phenyls, benzyl, and heterocycles. In certain embodiments R⁶ is selected from phenyl and benzyl. In certain embodiments the compound is disposed in a pharmaceutically-acceptable carrier to form a composition. In at least certain embodiments, the present disclosure is directed to a method of treating a subject for at least one of insulin resistance, obesity, Type 2 diabetes, Nonalcoholic fatty liver disease (NAFLD), and peroxisome proliferator-activated receptor gamma (PPARγ) S273 phosphorylation, by administering any one or more of the above compounds having chemical structure (I) to the subject.

In certain embodiments, the present disclosure is directed to compound having the chemical structure (II):

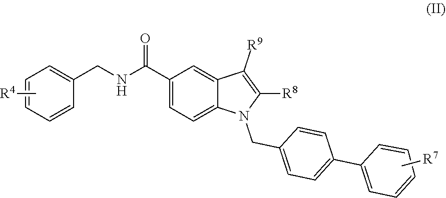

wherein, R⁴ is one or independently two of the group consisting of H, OH, alkyl, alkoxy, OCH₃, NH₂, SO₂, CF₃, COOH, SO₂NH₂, CN, CONH₂, phenyl, substituted phenyls, benzyl, and heterocycles; R⁷ is selected from the group consisting of H, COOH, NH, SO₂NH, B(OH)₂, CF₃, and heterocycle; R⁸ is selected from the group consisting of H, D, alkyl, cycloalkyl, aryl, substituted aryl, and heterocycle; and R⁹ is selected from the group consisting of H, D, alkyl, cycloalkyl, aryl, substituted aryl, and heterocycle. In certain embodiments, R⁴ comprises a single group selected from H, OH, alkyl, alkoxy, OCH₃, NH₂, SO₂, CF₃, COOH, SO₂NH₂, CN, CONH₂, phenyl, substituted phenyls, benzyl, and heterocycle. In certain embodiments, R⁴ comprises a pair of groups selected independently from H, OH, alkyl, alkoxy, OCH₃, NH₂, SO₂, CF₃, COOH, SO₂NH₂, CN, CONH₂, phenyl, substituted phenyls, benzyl, and heterocycle. In certain embodiments, at least one of or both of R⁸ and R⁹≠H or D. In certain embodiments, the compound is disposed in a pharmaceutically-acceptable carrier to form a composition. In at least certain embodiments, the present disclosure is directed to a method of treating a subject for at least one of insulin resistance, obesity, Type 2 diabetes, Nonalcoholic fatty liver disease (NAFLD), and peroxisome proliferator-activated receptor gamma (PPARγ) S273 phosphorylation, by administering any one or more of the above compounds having chemical structure (II) to the subject.

In certain embodiments, the present disclosure is directed to compound having the chemical structure (III):

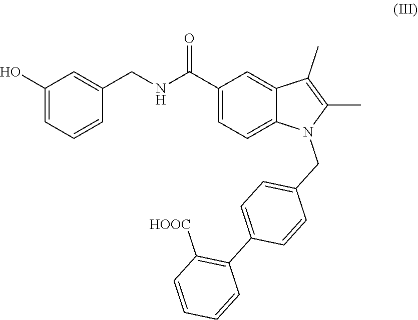 [SMILES: Cc1c(C)n(Cc2ccc(-c3ccccc3C(=O)O)cc2)c2ccc(C(=O)NCc3cccc(O)c3)cc12]

In certain embodiments, the compound is disposed in a pharmaceutically-acceptable carrier forming a composition. In at least certain embodiments, the present disclosure is directed to a method of treating a subject for at least one of insulin resistance, obesity, Type 2 diabetes, Nonalcoholic fatty liver disease (NAFLD), and peroxisome proliferator-activated receptor gamma (PPARγ) S273 phosphorylation, by administering any one or more of the above compounds having chemical structure (III) to the subject.

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure. Changes may be made in the formulations of the various compounds, compositions, and methods described herein, or in the steps or the sequence of steps of the methods described herein, without departing from the spirit and scope of the present disclosure. Further, while various embodiments of the present disclosure have been described in several particular claims below, it is not intended that the present disclosure be limited to these particular embodiments.

What is claimed is:

1. A compound comprising chemical structure (I): 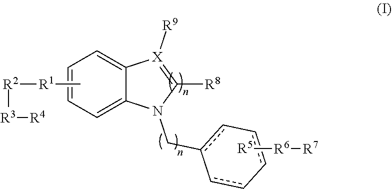
wherein
n₁=1 or 2;
n₂=1 or 2;
X═C or N;
R¹ is selected from the group consisting of CH₂, C═O, and NH;
R² is selected from the group consisting of CH₂, C═O, NH;
R³ is selected from the group consisting of alkyl, aryl, substituted aryl, and heterocycle;
R⁴ is one or independently two of the group consisting of H, OH, alkyl, alkoxy, OCH₃, NH₂, SO₂, CF₃, COOH, SO₂NH₂, CN, CONH₂, phenyl, substituted phenyls, benzyl, and heterocycles;
R⁵ is selected from the group consisting of alkyl, cycloalkyl, aryl, and heterocycle;
R⁶ is selected from the group consisting of alkyl, cycloalkyl, aryl, CH₂, NH, O, OCH₃, SH, SCH₃;
R⁷ is selected from the group consisting of H, COOH, NH, SO₂NH, B(OH)₂, CF₃, and heterocycle;
R⁸ is selected from the group consisting of H, D, alkyl, cycloalkyl, aryl, substituted aryl, and heterocycle; and
R⁹ is selected from the group consisting of H, D, alkyl, cycloalkyl, aryl, substituted aryl, and heterocycle.

2. The compound of claim 1, wherein at least one of or both of R⁸ and R⁹≠H or D.

3. The compound of claim 1, wherein R³ is selected from phenyl, and benzyl.

4. The compound of claim 1, wherein R⁴ comprises a single group selected from H, OH, alkyl, alkoxy, OCH₃, NH₂, SO₂, CF₃, COOH, SO₂NH₂, CN, CONH₂, phenyl, substituted phenyls, benzyl, and heterocycles.

5. The compound of claim 1, wherein R⁴ comprises a pair of groups selected independently from H, OH, alkyl, alkoxy, OCH₃, NH₂, SO₂, CF₃, COOH, SO₂NH₂, CN, CONH₂, phenyl, substituted phenyls, benzyl, and heterocycles.

6. The compound of claim 1, wherein R⁶ is selected from phenyl, and benzyl.

7. The compound of claim 1, disposed in a pharmaceutically-acceptable carrier.

8. The compound of claim 1, comprising the chemical structure (II): 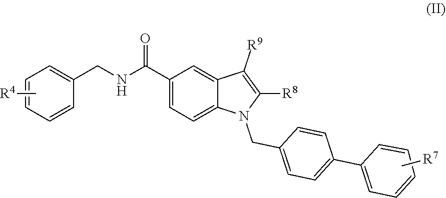
wherein
R⁴ is one or independently two of the group consisting of H, OH, alkyl, alkoxy, OCH₃, NH₂, SO₂, CF₃, COOH, SO₂NH₂, CN, CONH₂, phenyl, substituted phenyls, benzyl, and heterocycles;
R⁷ is selected from the group consisting of H, COOH, NH, SO₂NH, B(OH)₂, CF₃, and heterocycle;
R⁸ is selected from the group consisting of H, D, alkyl, cycloalkyl, aryl, substituted aryl, and heterocycle; and
R⁹ is selected from the group consisting of H, D, alkyl, cycloalkyl, aryl, substituted aryl, and heterocycle.

9. The compound of claim 8, wherein R⁴ comprises a single group selected from H, OH, alkyl, alkoxy, OCH₃, NH₂, SO₂, CF₃, COOH, SO₂NH₂, CN, CONH₂, phenyl, substituted phenyls, benzyl, and heterocycle.

10. The compound of claim 8, wherein R⁴ comprises a pair of groups selected independently from H, OH, alkyl, alkoxy, OCH₃, NH₂, SO₂, CF₃, COOH, SO₂NH₂, CN, CONH₂, phenyl, substituted phenyls, benzyl, and heterocycle.

11. The compound of claim 8, wherein at least one of or both of R⁸ and R⁹≠H or D.

12. The compound of claim 8 disposed in a pharmaceutically-acceptable carrier.

13. A compound comprising chemical structure (III):

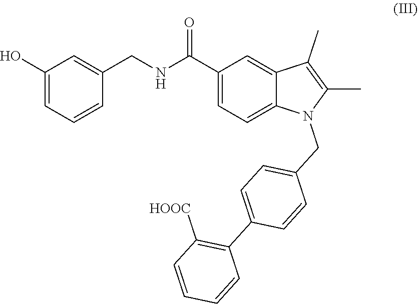 [SMILES: Cc1c(C)n(Cc2ccc(-c3ccccc3C(=O)O)cc2)c2ccc(C(=O)NCc3cccc(O)c3)cc12]

14. The compound of claim 13 disposed in a pharmaceutically-acceptable carrier.
